(19) United States
(12) Patent Application Publication
(10) Pub. No.: US 2023/0310627 A1
(43) Pub. Date: Oct. 5, 2023

(54) COMBINATION THERAPY WITH ANTI-TROP-2 ANTIBODIES AND PARP INHIBITORS

(71) Applicant: The General Hospital Corporation, Boston, MA (US)

(72) Inventors: Aditya BARDIA, Lexington, MA (US); Leif W ELLISEN, Newton, MA (US)

(21) Appl. No.: 18/056,038

(22) Filed: Nov. 16, 2022

Related U.S. Application Data
(60) Provisional application No. 63/329,505, filed on Apr. 11, 2022.
(60) Provisional application No. 63/280,401, filed on Nov. 17, 2021.

(51) Int. Cl. A61K 47/68; A61K 31/5025; A61P 35/04; A61K 45/06

(52) U.S. Cl. CPC A61K 47/6803; A61K 47/6851; A61K 31/5025; A61P 35/04; A61K 45/06; A61K 2039/545

(57) ABSTRACT

The present disclosure relates to a combination therapy with an anti-Trophoblast cell surface antigen 2 (Trop-2) antibody drug conjugate (anti-Trop-2 ADC) and a poly (ADP-ribose) polymerase inhibitor (PARPi) using a staggered dosing schedule. The ADC preferably incorporates an inhibitor of type I topoisomerase, such as SN-38 or DXd. The ADC is preferably administered prior to the PARPi in each cycle of the staggered dosing schedule. The combination therapy can reduce solid tumors in size, reduce or eliminate metastases and is effective to treat cancers resistant to standard therapies. The schedules and dosages show efficacy against tumors, while exhibiting only manageable toxicity to normal tissues. Use of staggered dosing of an anti-Trop-2 ADC and a PARPi can reduce toxicity, for example relative to alternative dosing schedules involving daily administrations of the PARPi on each day of a cycle.

### CROSS REFERENCE TO RELATED APPLICATIONS

This application claims benefit to U.S. Provisional Application No. 63/280,401, filed Nov. 17, 2021, and U.S. Provisional Application No. 63/329,505, filed Apr. 11, 2022, each of which is incorporated by reference herein in their entirety.

### FIELD

The present disclosure relates to combination therapy with anti-Trophoblast cell surface antigen 2 (Trop-2) antibody drug conjugates (ADCs) and PARP (poly ADP ribose polymerase) inhibitors, for treating Trop-2 expressing cancers such as triple-negative breast cancer (TNBC).

### BACKGROUND

Because of the overlapping toxicities between PARP inhibitors and anti-Trop-2 ADCs conjugated to agents that induce DNA strand breaks, such as topoisomerase inhibitors, a need exists for more effective techniques of administration that reduce the toxicity to normal tissues of such combination therapies, while retaining anti-tumor efficacy.

### SUMMARY

The present disclosure provides combination therapy for Trop-2 expressing cancers, using an anti-Trop-2 antibody-drug conjugate (ADC) combined with a PARP inhibitor (PARPi) in a staggered dosing schedule. In some embodiments the method of treating cancer comprises a) administering to a human subject with a cancer that expresses Trop-2 an antibody-drug conjugate (ADC) that binds to Trop-2, wherein the drug component of the ADC is a topoisomerase inhibitor; b) administering to the subject a Poly(ADP-ribose) polymerase inhibitor (PARPi), wherein the ADC and the PARPi are administered using a staggered dosing schedule. In some embodiments the ADC is sacituzumab govitecan. In some embodiments the PARPi is talazoparib. In some embodiments the ADC is administered on days 1 and 8 of a 21-day cycle. In some embodiments the PARPi is administered on days 15 to 21 of the 21-day cycle. In some embodiments the ADC is administered at a dosage of 8 mg/kg to 10 mg/kg (e.g., 10 mg/kg). In some embodiments the PARPi is administered at a dosage of 0.5 mg to 1.0 mg (e.g., 1.0 mg). In some embodiments the combination therapy methods provided herein, using a staggered dosing schedule, can reduce toxicity observed in human subjects relative to alternative combination treatment methods not using a staggered dosing schedule (e.g., alternative methods involving daily administrations of a PARPi on each day of a cycle).

A number of PARP inhibitors are known in the art and may be utilized in the subject combination therapy, including but not limited to olaparib, talazoparib, rucaparib, veliparib, niraparib, pamiparib, CEP 9722, E7016, CEP-8983 and 3-aminobenzamide (see, e.g., Rouleau et al., 2010, Nat Rev Cancer 10:293-301, Bao et al., 2015, Oncotarget [Epub ahead of print, Sep. 22, 2015]). Any such known PARP inhibitor may be used in combination with an anti-Trop-2 ADC. Preferably, the PARP inhibitor is one that exhibits synergistic effects when used in combination with the ADC. More preferably, the PARP inhibitor is olaparib, talazoparib or rucaparib. Most preferably, the PARPi is talazoparib.

Exemplary anti-Trop-2 antibodies that may be utilized include, but are not limited to, hRS7 (U.S. Pat. No. 7,238,785) and dapotomab (hTINA1, U.S. Pat. Nos. 9,850,312; 10,227,417; 11,008,398). In a preferred embodiment, the anti-Trop-2 antibody may be a humanized RS7 antibody (see, e.g., U.S. Pat. No. 7,238,785, incorporated herein by reference in its entirety). However, as discussed below other anti-Trop-2 antibodies are known and may be used.

Preferably, the antibody or fragment thereof is linked to at least one chemotherapeutic moiety; preferably 1 to about 5 drug moieties; more preferably 6 to about 12 drug moieties, most preferably about 6 to 8 drug moieties.

Various embodiments may concern use of the subject methods and compositions to treat a cancer that expresses Trop-2, including but not limited to carcinomas, melanomas, sarcomas, gliomas, bone and skin cancers. The carcinomas may include carcinomas of the oral cavity, esophagus, gastrointestinal tract, pulmonary tract, lung, stomach, colon, rectum, breast, ovary, prostate, uterus, endometrium, cervix, pancreas, bone, brain, connective tissue, thyroid, liver, gall bladder, urinary bladder (urothelial), kidney, skin, central nervous system and testes.

In certain embodiments, the combination of ADC and PARPi may be used in conjunction with a standard anti-cancer treatment, such as surgery, radiation therapy, chemotherapy, immunotherapy with naked antibodies, including checkpoint-inhibiting antibodies, other drug-conjugated antibodies, radioimmunotherapy, immunomodulators, and the like. These combination therapies can allow lower doses of each therapeutic agent to be given in such combinations, thus reducing certain severe side effects, and potentially reducing the courses of therapy required. When there is no or minimal overlapping toxicity, full doses of each agent can also be given.

### BRIEF DESCRIPTION OF THE DRAWINGS

FIG. 1A shows a graph illustrating the in vitro cytotoxicity of sacituzumab govitecan (SG)+talazoparib (TZP) combination treatments administered on a staggered schedule on MDA-MB-468 breast cancer cells.

FIG. 1B shows results of a western blot for topoisomerase 1 (TOP1) covalently bound to DNA, illustrating the stabilizing effect of SG+TZP combination treatments administered on a staggered schedule on TOP1 cleavage complex in MDA-MB-468 breast cancer cells in vitro.

FIG. 1C shows microscopic fluorescence images visualizing the stabilization of TOP1 cleavage complexes (TOP1CC) by SG+TZP combination treatments administered on a staggered schedule in MDA-MB-468 breast cancer cells in vitro.

FIG. 1D shows a bar graph illustrating results of a quantitative analysis of TOP1CC foci per cell based on the fluorescence images of FIG. 1C.

FIG. 1E shows FACS graphs illustrating phosphorylation of Ser139 of histone variant H2AX (gH2AX, also known as gamma-H2AX, Y-axis), an early cellular response to the induction of DNA double-strand breaks, in MDA-MB-468 breast cancer cells following SG treatment combined with 24 h medium wash out or 24 h TZP treatment, plus SG alone and TZP alone treatment controls.

FIG. 1F shows a bar graph depicting the quantification of FACS results shown in FIG. 1E.

FIG. 1G shows FACS graphs illustrating propidium iodide (PI) (Y-axis) and Annexin V (X-axis) staining, both apoptosis markers, in MDA-MB-468 breast cancer cells following SG treatment combined with 24 h or 48 h medium wash out or 24 h or 48 h TZP treatment, plus SG alone and TZP alone treatment controls.

FIG. 1H shows a bar graph depicting the quantification of FACS results shown in FIG. 1G.

FIG. 1I shows a graph illustrating the in vitro cytotoxicity of SG+TZP combination treatments administered on a staggered schedule on normal diploid cells (WI-38).

FIG. 1J shows a graph illustrating the relative in vitro cytotoxicity of TZP treatments on MDA-MB-468 breast cancer and WI-38 normal diploid cells.

FIG. 1K shows a graph illustrating the in vitro cytotoxicity of SG+TZP combination treatments administered on a staggered schedule on HCC1806 breast cancer cells.

FIG. 1L shows results of a western blot for TOP1 covalently bound to DNA, illustrating the stabilizing effect of SG+TZP combination treatments administered on a staggered schedule on TOP1 cleavage complex in HCC1806 breast cancer cells in vitro.

FIG. 2A shows a graphic representation of a clinical trial design to compare SG+TZP combination treatments administered on either continuous or staggered dosing schedules to human patients with metastatic triple-negative breast cancer (mTNBC).

FIG. 2B shows a graph illustrating efficacy results for the clinical trial presented in FIG. 2A for patients treated on a continuous dosing schedule. The top left panel is a graph showing change from baseline (%) and objective response rate (ORR; RECIST V.1.1) information for patients treated on a continuous dosing schedule in Cohort 1 and Cohort 1A, the bottom left panel shows information regarding biomarkers and mutations for the indicated patients, the top right panel shows a key for the biomarker and mutation information, and the bottom right panel shows a key for the cohort for the indicated patients for the data in FIG. 2B and FIG. 2C.

FIG. 2C shows a graph illustrating efficacy results for the clinical trial presented in FIG. 2A for patients treated on a staggered dosing schedule. The top panel shows is a graph showing change from baseline (%) and ORR (RECIST V.1.1) information for patients treated on a staggered dosing schedule in Cohort 1B, Cohort 4A, Cohort 3B, and Cohort 4B, and the bottom panel shows information regarding biomarkers and mutations for the indicated patients. The keys in the top right and bottom right panels of FIG. 2B also apply to FIG. 2C.

FIG. 3A shows a schematic illustrating exposure time courses for SN38 in normal and tumor tissues following systemic SG administration to a subject during a 7 day dosing cycle, and the relative timing of PARPi administrations on a staggered dosing schedule.

FIG. 3B shows a bar graph illustrating relative frequencies of dose-limiting toxicities (DLTs) observed in human mTNBC patients during a clinical SG/TZP combination trial on a concurrent and sequential (staggered) dosing schedule.

FIG. 3C shows a bar graph illustrating relative frequencies of neutropenia, anemia, thrombocytopenia, nausea, and diarrhea observed in human patients during a clinical SG/TZP combination trial on a concurrent and staggered dosing schedule.

FIG. 4A shows progression-free survival curves for human mTNBC patients receiving SG/TZP combination treatments on a continuous or sequential (staggered) dosing schedule (IMMU=SG).

FIG. 4B shows immunohistochemistry images of tumor biopsies obtained from an exemplary human mTNBC patient pre-treatment and post-SG/TZP treatment on a sequential (staggered) dosing schedule, absence or presence of g-H2AX biomarker expression is highlighted in boxed areas.

### DETAILED DESCRIPTION

### Definitions

The following definitions are provided to facilitate understanding of the claimed subject matter. Terms that are not expressly defined herein are used in accordance with their plain and ordinary meanings.

Unless otherwise specified, “a” or “an” means “one or more.”

The term “about” is used herein to mean plus or minus ten percent (10%) of a value. For example, “about 100” refers to any number between 90 and 110.

An “antibody,” as used herein, refers to a full-length (i.e., naturally occurring) immunoglobulin molecule (e.g., an IgG antibody) or an antigen-binding portion of an immunoglobulin molecule, such as an antibody fragment. An antibody or antibody fragment may be conjugated or otherwise derivatized within the scope of the claimed subject matter. Such antibodies include but are not limited to IgG1, IgG2, IgG3, IgG4 (and IgG4 subforms), as well as IgA isotypes. As used below, the abbreviation “Mab” may be used interchangeably to refer to an antibody, antibody fragment, monoclonal antibody or multispecific antibody.

An “antibody fragment” is a portion of an antibody such as F(ab′)₂, F(ab)₂, Fab′, Fab, Fv, scFv (single chain Fv), single domain antibodies (DAB s or VHHs) and the like, including the half-molecules of IgG4 cited above (van der Neut Kolfschoten et al. (Science 2007; 317(14 September):1554-1557). Regardless of structure, an antibody fragment of use binds with the same antigen that is recognized by the intact antibody. The term “antibody fragment” also includes synthetic or genetically engineered proteins that act like an antibody by binding to a specific antigen to form a complex. For example, antibody fragments include isolated fragment consisting of the variable regions, such as the “Fv” fragments consisting of the variable regions of the heavy and light chains and recombinant single chain polypeptide molecules in which light and heavy variable regions are connected by a peptide linker (“scFv proteins”). The fragments may be constructed in different ways to yield multivalent and/or multispecific binding forms.

A “naked antibody” is generally an entire antibody that is not conjugated to a therapeutic agent. A naked antibody may exhibit therapeutic and/or cytotoxic effects, for example by Fc-dependent functions, such as complement fixation (CDC) and ADCC (antibody-dependent cell cytotoxicity). However, other mechanisms, such as apoptosis, anti-angiogenesis, anti-metastatic activity, anti-adhesion activity, inhibition of heterotypic or homotypic adhesion, and interference in signaling pathways, may also provide a therapeutic effect. Naked antibodies include polyclonal and monoclonal antibodies, naturally occurring or recombinant antibodies, such as chimeric, humanized or human antibodies and fragments thereof. As defined herein, “naked” is synonymous with “unconjugated,” and means not linked or conjugated to a therapeutic agent.

A “chimeric antibody” is a recombinant protein that contains the variable domains of both the heavy and light antibody chains, including the complementarity determining regions (CDRs) of an antibody derived from one species, preferably a rodent antibody, more preferably a murine antibody, while the constant domains of the antibody molecule are derived from those of a human antibody.

A “humanized antibody” is a recombinant protein in which the CDRs from an antibody from one species; e.g., a murine antibody, are transferred from the heavy and light variable chains of the murine antibody into human heavy and light variable domains (framework regions). The constant domains of the antibody molecule are derived from those of a human antibody. In some cases, specific residues of the framework region of the humanized antibody, particularly those that are touching or close to the CDR sequences, may be modified, for example replaced with the corresponding residues from the original murine, rodent, subhuman primate, or other antibody.

A “human antibody” is an antibody obtained, for example, from transgenic mice that have been “engineered” to produce human antibodies in response to antigenic challenge. In this technique, elements of the human heavy and light chain loci are introduced into strains of mice derived from embryonic stem cell lines that contain targeted disruptions of the endogenous heavy chain and light chain loci. The transgenic mice can synthesize human antibodies specific for various antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. Phage display can be performed in a variety of formats, for their review, see e.g., Johnson and Chiswell, Current Opinion in Structural Biology 3:5564-571 (1993). Human antibodies may also be generated by in vitro activated B cells. See U.S. Pat. Nos. 5,567,610 and 5,229,275, the Examples section of each of which is incorporated herein by reference.

A “therapeutic agent” is an atom, molecule, or compound that is useful in the treatment of a disease. Examples of therapeutic agents include, but are not limited to, antibodies, antibody fragments, ADCs, drugs, cytotoxic agents, pro-apoptotic agents, toxins, nucleases (including DNAses and RNAses), hormones, immunomodulators, chelators, photoactive agents or dyes, radionuclides, oligonucleotides, interference RNA, siRNA, RNAi, anti-angiogenic agents, chemotherapeutic agents, cytokines, chemokines, prodrugs, enzymes, binding proteins or peptides or combinations thereof.

An “immunomodulator” is a therapeutic agent that when present, alters, suppresses or stimulates the body's immune system. Typically, an immunomodulator of use stimulates immune cells to proliferate or become activated in an immune response cascade, such as macrophages, dendritic cells, B-cells, and/or T-cells. However, in some cases an immunomodulator may suppress proliferation or activation of immune cells. An example of an immunomodulator as described herein is a cytokine, which is a soluble small protein of approximately 5-20 kDa that is released by one cell population (e.g., primed T-lymphocytes) on contact with specific antigens, and which acts as an intercellular mediator between cells. As the skilled artisan will understand, examples of cytokines include lymphokines, monokines, interleukins, and several related signaling molecules, such as tumor necrosis factor (TNF) and interferons. Chemokines are a subset of cytokines. Certain interleukins and interferons are examples of cytokines that stimulate T cell or other immune cell proliferation. Exemplary interferons include interferon-α, interferon-β, interferon-γ and interferon-λ.

Anti-Trop-2 Antibodies

The subject ADCs include at least one antibody or fragment thereof that binds to human Trop-2. In a specific preferred embodiment, the anti-Trop-2 antibody may be a humanized RS7 antibody (see, e.g., U.S. Pat. No. 7,238,785, incorporated herein by reference in its entirety).

The RS7 antibody was a murine IgG1 raised against a crude membrane preparation of a human primary squamous cell lung carcinoma (Stein et al., Cancer Res. 50: 1330, 1990). The RS7 antibody recognizes a 46-48 kDa glycoprotein, characterized as cluster 13 (Stein et al., Int. J. Cancer Supp. 8:98-102, 1994). The antigen was designated as EGP-1 (epithelial glycoprotein-1), also referred to as Trop-2.

Trop-2 is a type-I transmembrane protein and has been cloned from both human (Fornaro et al., Int J Cancer 1995; 62:610-8) and mouse cells (Sewedy et al., Int J Cancer 1998; 75:324-30). In addition to its role as a tumor-associated calcium signal transducer (Ripani et al., Int J Cancer 1998; 76:671-6), the expression of human Trop-2 was shown to be necessary for tumorigenesis and invasiveness of colon cancer cells, which could be effectively reduced with a polyclonal antibody against the extracellular domain of Trop-2 (Wang et al., Mol Cancer Ther 2008; 7:280-5).

The growing interest in Trop-2 as a therapeutic target for solid cancers (Cubas et al., Biochim Biophys Acta 2009; 1796:309-14) is attested by further reports that documented the clinical significance of overexpressed Trop-2 in breast (Huang et al., Clin Cancer Res 2005; 11:4357-64), colorectal (Ohmachi et al., Clin Cancer Res 2006; 12:3057-63; Fang et al., Int J Colorectal Dis 2009; 24:875-84), and oral squamous cell (Fong et al., Modern Pathol 2008; 21:186-91) carcinomas. Prostate basal cells expressing high levels of Trop-2 are enriched for in vitro and in vivo stem-like activity (Goldstein et al., Proc Natl Acad Sci USA 2008; 105:20882-7).

Flow cytometry and immunohistochemical staining studies have shown that the RS7 MAb detects antigen on a variety of tumor types, with limited binding to normal human tissue (Stein et al., 1990). Trop-2 is expressed primarily by carcinomas such as carcinomas of the lung, stomach, urinary bladder (urothelium), breast, ovary, uterus, and prostate. Localization and therapy studies using radiolabeled murine RS7 MAb in animal models have demonstrated tumor targeting and therapeutic efficacy (Stein et al., 1990; Stein et al., 1991).

Strong RS7 staining has been demonstrated in tumors from the lung, breast, bladder, ovary, uterus, stomach, and prostate (Stein et al., Int. J. Cancer 55:938, 1993). The lung cancer cases comprised both squamous cell carcinomas and adenocarcinomas (Stein et al., Int. J. Cancer 55:938, 1993). Both cell types stained strongly, indicating that the RS7 antibody does not distinguish between histologic classes of non-small-cell carcinoma of the lung.

The RS7 MAb is rapidly internalized into target cells (Stein et al., 1993). Internalization of ADCs has been described as a major factor in anti-tumor efficacy (Yang et al., Proc. Nat'l Acad. Sci. USA 85: 1189, 1988). However, the CL2A linker in SG also allows slow spontaneous release of SN-38 prior to internalization, facilitating a bystander effect for tumor toxicity. Thus, the RS7 antibody and its drug conjugates exhibit several important properties for therapeutic applications.

While the hRS7 antibody is preferred, other anti-Trop-2 antibodies are known and/or publicly available and in alternative embodiments may be utilized in the subject ADCs. Anti-Trop-2 antibodies are commercially available from a number of sources and include LS-C126418, LS-C178765, LS-C126416, LS-C126417 (LifeSpan BioSciences, Inc., Seattle, WA); 10428-MM01, 10428-MM02, 10428-R001, 10428-R030 (Sino Biological Inc., Beijing, China); MR54 (eBioscience, San Diego, CA); sc-376181, sc-376746, Santa Cruz Biotechnology (Santa Cruz, CA); MM0588-49D6, (Novus Biologicals, Littleton, CO); ab79976, and ab89928 (ABCAM®, Cambridge, MA).

Other anti-Trop-2 antibodies have been disclosed in the patent literature. For example, the anti-Trop-2 antibody dapotomab (hTINA1) has been disclosed in U.S. Pat. Nos. 9,850,312, 10,227,417, and 11,008,398. U.S. Publ. No. 2013/0089872 discloses anti-Trop-2 antibodies K5-70 (Accession No. FERM BP-11251), K5-107 (Accession No. FERM BP-11252), K5-116-2-1 (Accession No. FERM BP-11253), T6-16 (Accession No. FERM BP-11346), and T5-86 (Accession No. FERM BP-11254), deposited with the International Patent Organism Depositary, Tsukuba, Japan. U.S. Pat. No. 5,840,854 disclosed the anti-Trop-2 monoclonal antibody BR110 (ATCC No. HB11698). U.S. Pat. No. 7,420,040 disclosed an anti-Trop-2 antibody produced by hybridoma cell line AR47A6.4.2, deposited with the IDAC (International Depository Authority of Canada, Winnipeg, Canada) as accession number 141205-05. U.S. Pat. No. 7,420,041 disclosed an anti-Trop-2 antibody produced by hybridoma cell line AR52A301.5, deposited with the IDAC as accession number 141205-03. U.S. Publ. No. 2013/0122020 disclosed anti-Trop-2 antibodies 3E9, 6G11, 7E6, 15E2, 18B1. Hybridomas encoding an anti-Trop-2 antibody were deposited with the American Type Culture Collection (ATCC), Accession Nos. PTA-12871 and PTA-12872. U.S. Pat. No. 8,715,662 discloses anti-Trop-2 antibodies produced by hybridomas deposited at the AID-ICLC (Genoa, Italy) with deposit numbers PD 08019, PD 08020 and PD 08021. U.S. Patent Application Publ. No. 20120237518 discloses anti-Trop-2 antibodies 77220, KM4097 and KM4590. U.S. Pat. No. 8,309,094 (Wyeth) discloses antibodies A1 and A3, identified by sequence listing. The Examples section of each patent or patent application cited above in this paragraph is incorporated herein by reference. Non-patent publication Lipinski et al. (1981, Proc Natl. Acad Sci USA, 78:5147-50) disclosed anti-Trop-2 antibodies 162-25.3 and 162-46.2.

Numerous anti-Trop-2 antibodies are known in the art and/or publicly available. As discussed below, methods for preparing antibodies against known antigens were routine in the art. The sequence of the human Trop-2 protein was also known in the art (see, e.g., GenBank Accession No. CAA54801.1). The person of ordinary skill, reading the instant disclosure in light of general knowledge in the art, would have been able to make and use the genus of anti-Trop-2 antibodies in the subject ADCs.

PARP Inhibitors

Poly-(ADP-ribose) polymerase (PARP) plays a key role in the DNA damage response and either directly or indirectly affects numerous DDR pathways, including BER, HR, NER, NHEJ and MMR (Gavande et al., 2016, Pharmacol Ther 160:65-83). A number of PARP inhibitors are known in the art, such as olaparib, talazoparib (BMN-673), rucaparib, veliparib, niraparib, pamiparib, CEP 9722, CEP-8983, E7016 and 3-aminobenzamide (see, e.g., Rouleau et al., 2010, Nat Rev Cancer 10:293-301, Bao et al., 2015, Oncotarget [Epub ahead of print, Sep. 22, 2015]). PARP inhibitors are known to exhibit synthetic lethality, for example in tumors with mutations in BRCA1/2. Olaparib has received FDA approval for treatment of ovarian cancer patients with mutations in BRCA1 or BRCA2. In addition to olaparib, other FDA-approved PARP inhibitors for ovarian cancer include nirapirib and rucaparib. Talazoparib has been approved for treatment of breast cancer with germline BRCA mutations and is in phase III trials for hematological malignancies and solid tumors and has reported efficacy in SCLC, ovarian, breast, and prostate cancers (Bitler et al., 2017, Gynecol Oncol 147:695-704). Veliparib is in phase III trials for advanced ovarian cancer, TNBC and NSCLC (see Wikipedia under “PARP_inhibitor”). Not all PARP inhibitors are dependent on BRCA mutation status and niraparib has been approved for maintenance therapy of recurrent platinum sensitive ovarian, fallopian tube or primary peritoneal cancer, independent of BRCA status (Bitler et al., 2017, Gynecol Oncol 147:695-704).

Any such known PARP inhibitor may be utilized in combination with an anti-Trop-2 ADC, such as sacituzumab govitecan or DS-1062 (datopotamab deruxtecan). Synthetic lethality and synergistic inhibition of tumor growth has been demonstrated for the combination of sacituzumab govitecan with olaparib, rucaparib and talazoparib in nude mice bearing TNBC xenografts (Cardillo et al., 2017, Clin Cancer Res 23:3405-15). The beneficial effects of combination therapy were observed independently of BRCA1/2 mutation status (Cardillo et al., 2017, Clin Cancer Res 23:3405-15).

Therapeutic Treatment

This disclosure is based, at least in part, on the recognition that antibody-mediated tumor-selective delivery of a TOP1 inhibitor, such as SN38, via an anti-Trop-2 antibody-drug-conjugate (anti-Trop-2), such as sacituzumab govitecan (SG), can enable sequential dosing of SG with a PARP inhibitor (e.g., talazoparib) in a staggered dosing schedule to enhance the therapeutic window and allow delivery of the combination with less toxicity.

In one aspect, provided herein is a method of treating cancer comprising administering to a human subject with a cancer that expresses Trop-2 a therapeutically effective amount of an ADC as described herein in combination with a PARP inhibitor using a staggered dosing schedule.

As used herein, the term “staggered dosing schedule” refers to a dosing schedule in which two or more different therapeutic agents are administered on different days of a dosing cycle. Generally, staggered dosing schedules, as used herein, are types of sequential dosing schedules and distinguishable from concurrent dosing schedules. In some embodiments the staggered dosing schedule comprises in each cycle a plurality of administrations of one or more of the two or more different therapeutic agents, with each administration occurring on a separate day. In some embodiments each administration of a plurality of administrations of a first therapeutic agent in a cycle occurs prior to the first administration of a second therapeutic agent in the cycle. In some embodiments of a staggered dosing schedule a first therapeutic agent (e.g., sacituzumab govitecan) is administered on days 1 and 8 of a 21-day cycle and a second therapeutic agent (e.g., talazoparib) is administered on days 15 to 21 of the 21-day cycle. An example of a dosing schedule that is not staggered is a schedule wherein a first therapeutic agent (e.g., sacituzumab govitecan) is administered on days 1 and 8 of a 21-day cycle and a second therapeutic agent (e.g., talazoparib) is administered on days 1 to 21 of the 21-day cycle.

In some embodiments the method of treating cancer comprises a) administering to a human subject with a cancer that expresses Trop-2 an antibody-drug conjugate (ADC) that binds to Trop-2, wherein the drug component of the ADC is a topoisomerase inhibitor; b) administering to the subject a Poly(ADP-ribose) polymerase inhibitor (PARPi), wherein the ADC and the PARPi are administered using a staggered dosing schedule. In some embodiments the topoisomerase inhibitor is an inhibitor of topoisomerase I. In some embodiments, the topoisomerase inhibitor is selected from the group consisting of SN-38, camptothecin, topotecan, irinotecan, belotecan, rubitecan, exatecan, deruxtecan (DXd), gimatecan, silatecan, idenoisoquinoline, a phenanthridine, and an indolocarbazole. In some embodiments, the anti-Trop-2 antibody component of the ADC is sacituzumab (hRS7) or datopotamab. In some embodiments the ADC comprises an anti-Trop-2 hRS7 antibody conjugated to an SN-38 topoisomerase inhibitor via a CL2A linker. In some embodiments the ADC is sacituzumab govitecan or datopotamab deruxtecan (DS-1062). In some embodiments the ADC is sacituzumab govitecan. In some embodiments the PARPi is selected from the group consisting of olaparib, talazoparib, rucaparib, veliparib, niraparib, pamiparib, CEP 9722, E7016, CEP-8983, and 3-aminobenzamide. In some embodiments the PARPi is talazoparib. In some embodiments the staggered dosing schedule comprises a 21-day cycle. In some embodiments the anti-Trop-2 ADC is administered on days 1 and 8 of the 21-day cycle. In some embodiments the PARPi is administered on days 15 to 21 of the 21-day cycle. In some embodiments the ADC is administered at a dosage of 8 mg/kg to 10 mg/kg. In some embodiments the ADC is administered at a dosage of 10 mg/kg. In some embodiments the PARPi is administered at a dosage of 0.5 mg/kg to 1.0 mg/kg. In some embodiments the PARPi is administered at a dosage of 1.0 mg/kg. In some embodiments the cancer is selected from the group consisting of colon cancer, stomach cancer, esophageal cancer, medullary thyroid cancer, kidney cancer, breast cancer, lung cancer, pancreatic cancer, urothelial cancer, ovarian cancer, uterine cancer, cervical cancer, testicular cancer, prostate cancer, liver cancer, skin cancer, bone cancer, head and neck cancer, brain cancer, glioblastoma, rectal cancer, and melanoma. In some embodiments the cancer is selected from the group consisting of triple-negative breast cancer, HR+/HER2− breast cancer, HER2-low breast cancer, ovarian cancer, endometrial cancer, urothelial cancer, non-small-cell lung cancer, small-cell lung cancer and colorectal cancer. In some embodiments the subject does not exhibit a mutation in BRCA1 or BRCA2 (e.g., as determined by an FDA-approved companion diagnostic test for the detection of BRCA mutations). In some embodiments the cancer is locally advanced, e.g., locally advanced breast cancer (e.g., locally advanced TNBC). In some embodiments the cancer is metastatic, e.g., metastatic breast cancer (e.g., metastatic TNBC). In some embodiments the cancer is HER2-negative metastatic breast cancer with deleterious or suspected deleterious germline breast cancer susceptibility gene (BRCA)-mutated (gBRCAm). In some embodiments the cancer is metastatic triple negative breast cancer (mTNBC). In some embodiments the staggered dosing schedule reduces the toxicity of the treatment to normal tissues (e.g., relative to a dosing schedule comprising a daily administration of the PARPi on each day of a cycle). In some embodiments the staggered dosing schedule reduces the incidence of neutropenia (e.g., relative to a dosing schedule comprising a daily administration of the PARPi on each day of a cycle). In some embodiments the staggered dosing schedule reduces the incidence of adverse events (e.g., relative to a dosing schedule comprising a daily administration of the PARPi on each day of a cycle). In some embodiments, the adverse event is selected from the group consisting of neutropenia, anemia, thrombocytopenia, nausea, and diarrhea. In some embodiments, the staggered dosing schedule improves progression free survival relative to a continuous dosing schedule. In some embodiments, the staggered dosing schedule improves overall survival relative to a continuous dosing schedule. In some embodiments the cancer is metastatic (e.g., metastatic breast cancer) and the treatment reduces in size or eliminates the metastases. In some embodiments, the subject has received at least two prior therapies for metastatic disease. In some embodiments the cancer is refractory to at least one other therapy but responds to the combination of ADC and PARPi. In some embodiments, the method further comprises administering to the subject one or more additional therapeutic modalities selected from the group consisting of unconjugated antibodies, radiolabeled antibodies, drug-conjugated antibodies, toxin-conjugated antibodies, gene therapy, chemotherapy, therapeutic peptides, cytokine therapy, oligonucleotides, localized radiation therapy, surgery and interference RNA therapy. In some embodiments the therapeutic modality comprises treatment with an agent selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celebrex, chlorambucil, cisplatin (CDDP), Cox-2 inhibitors, irinotecan (CPT-11), SN-38, carboplatin, cladribine, camptothecans, cyclophosphamide, crizotinib, cytarabine, dacarbazine, dasatinib, dinaciclib, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, flavopiridol, floxuridine (FUdR), 3′,5′-O-dioleoyl-FudR (FUdR-dO), fludarabine, flutamide, farnesyl-protein transferase inhibitors, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD1839.

In some embodiments the method of treating cancer provided herein comprises a) administering to a human subject with metastatic breast cancer sacituzumab govitecan at a daily dosage of 10 mg/kg on days 1 and 8 of a 21-day dosing cycle and administering talazoparib at a daily dosage of 1 mg on each of days 15 to 21 of the 21-day dosing cycle.

In some embodiments the method of treating cancer provided herein comprises a) administering to a human subject with metastatic triple negative breast cancer (mTNBC) sacituzumab govitecan at a daily dosage of 10 mg/kg on days 1 and 8 of a 21-day dosing cycle and administering talazoparib at a daily dosage of 1 mg on each of days 15 to 21 of the 21-day dosing cycle.

In some embodiments, the methods provided herein comprise administering sacituzumab govitecan and talazoparib to a human metastatic breast cancer patient on a staggered dosing schedule, wherein sacituzumab govitecan is administered on days 1 and 8 of a 21 day cycle at a dosage of 8 mg/kg to 10 mg/kg, and talazoparib is administered on days 15 to 21 of the 21 day cycle at a dosage of 0.5 mg/kg to 1.0 mg/kg. In some embodiments sacituzumab govitecan is administered at a dosage of 8 mg/kg. In some embodiments, sacituzumab govitecan is administered at a dosage of 10 mg/kg. In some embodiments, talazoparib is administered at a dosage of 0.5 mg/kg. In some embodiments talazoparib is administered at a dosage of 1.0 mg/kg.

In some embodiments, the methods provided herein comprise administering sacituzumab govitecan and talazoparib to a human metastatic TNBC patient on a staggered dosing schedule, wherein sacituzumab govitecan is administered on days 1 and 8 of a 21 day cycle at a dosage of 8 mg/kg to 10 mg/kg, and talazoparib is administered on days 15 to 21 of the 21 day cycle at a dosage of 0.5 mg/kg to 1.0 mg/kg. In some embodiments, sacituzumab govitecan is administered at a dosage of 8 mg/kg. In some embodiments, sacituzumab govitecan is administered at a dosage of 10 mg/kg. In some embodiments, talazoparib is administered at a dosage of 0.5 mg/kg. In some embodiments talazoparib is administered at a dosage of 1.0 mg/kg.

Diseases that may be treated with the combination therapy described herein include, but are not limited to adenocarcinomas of endodermally-derived digestive system epithelia, cancers such as breast cancer and non-small cell lung cancer, and other carcinomas, sarcomas, glial tumors, etc. In particular, antibodies against a Trop-2 antigen, produced by or associated with a malignant solid tumor, e.g., a gastrointestinal, stomach, colon, esophageal, liver, lung, breast, pancreatic, hepatocellular, renal, urothelial, prostate, ovarian, endometrial, cervical, testicular, brain, head and neck or bone tumor, a sarcoma or a melanoma, are advantageously used. Such therapeutics can be given once or repeatedly, depending on the disease state and tolerability of the conjugate, and can also be used optionally in combination with other therapeutic modalities, such as surgery, external radiation, radioimmunotherapy, immunotherapy, chemotherapy, antisense therapy, interference RNA therapy, gene therapy, and the like. Use of an anti-Trop-2 ADC in combination with a PARP inhibitor does not exclude further combination with additional therapeutic modalities, as discussed herein. Each combination will be adapted to the tumor type, stage, patient condition and prior therapy, and other factors considered by the managing physician.

As used herein, the term “subject” refers to any animal (i.e., vertebrates and invertebrates) including, but not limited to mammals, including humans. It is not intended that the term be limited to a particular age or sex. Thus, adult and newborn subjects, as well as fetuses, whether male or female, are encompassed by the term. In preferred embodiments, the subject is a human subject and doses disclosed herein are for adult humans, but can be adjusted for other mammals, as well as children. Where an ADC is administered to a human subject, the person of ordinary skill will realize that the target antigen to which the ADC binds will be a human antigen, such as human Trop-2.

Therapeutic agents of use in combination with the ADC and PARPi described herein also include, for example, chemotherapeutic drugs such as vinca alkaloids, anthracyclines, epipodophyllotoxins, taxanes, antimetabolites, tyrosine kinase inhibitors, Bruton tyrosine kinase inhibitors, microtubule inhibitors, PARP inhibitors, other DDR inhibitors, PI3K inhibitors, alkylating agents, antibiotics, Cox-2 inhibitors, antimitotics, antiangiogenic and proapoptotic agents, particularly doxorubicin, methotrexate, taxol, other camptothecins, and others from these and other classes of anticancer agents, and the like. Other cancer chemotherapeutic drugs include nitrogen mustards, alkyl sulfonates, nitrosoureas, triazenes, folic acid analogs, pyrimidine analogs, purine analogs, platinum coordination complexes, hormones, and the like. Suitable chemotherapeutic agents are described in REMINGTON'S PHARMACEUTICAL SCIENCES, 19th Ed. (Mack Publishing Co. 1995), and in GOODMAN AND GILMAN'S THE PHARMACOLOGICAL BASIS OF THERAPEUTICS, 7th Ed. (MacMillan Publishing Co. 1985), as well as revised editions of these publications. Other suitable chemotherapeutic agents, such as experimental drugs, are known to those of skill in the art.

Exemplary drugs of use include, but are not limited to, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatin, Cox-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, doxorubicin, 2-pyrrolinodoxorubicine (2PDOX), cyano-morpholino doxorubicin, doxorubicin glucuronide, epirubicin glucuronide, erlotinib, estramustine, epipodophyllotoxin, erlotinib, entinostat, estrogen receptor binding agents, etoposide, etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosourea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide, transplatin, thalidomide, thioguanine, thiotepa, teniposide, topotecan, uracil mustard, vatalanib, vinorelbine, vinblastine, vincristine, vinca alkaloids and ZD1839. Such agents may be part of the conjugates described herein or may alternatively be administered in combination with the described conjugates, either prior to, simultaneously with or after the conjugate. Alternatively, one or more therapeutic naked antibodies as are known in the art may be used in combination with the described ADCs and/or PARPi.

In preferred embodiments, a therapeutic agent to be used in combination with an ADC and/or PARPi is a microtubule inhibitor, such as a vinca alkaloid, a taxane, a maytansinoid or an auristatin. Exemplary known microtubule inhibitors include paclitaxel, vincristine, vinblastine, mertansine, epothilone, docetaxel, discodermolide, combrestatin, podophyllotoxin, CI-980, phenylahistins, steganacins, curacins, 2-methoxy estradiol, E7010, methoxy benzenesuflonamides, vinorelbine, vinflunine, vindesine, dolastatins, spongistatin, rhizoxin, tasidotin, halichondrins, hemiasterlins, cryptophycin 52, MMAE and eribulin mesylate.

In an alternative preferred embodiment, a therapeutic agent used in combination with an ADC and/or PARPi is a Bruton kinase inhibitor, such as such as ibrutinib (PCI-32765), PCI-45292, CC-292 (AVL-292), ONO-4059, GDC-0834, LFM-A13 or RN486.

In another alternative, a therapeutic agent used in combination with an ADC and/or PARPi is a PI3K inhibitor, such as idelalisib, Wortmannin, demethoxyviridin, perifosine, PX-866, IPI-145 (duvelisib), BAY 80-6946, BEZ235, RP6530, TGR1202, SF1126, INK1117, GDC-0941, BKM120, XL147, XL765, Palomid 529, GSK1059615, ZSTK474, PWT33597, IC87114, TG100-115, CAL263, PI-103, GNE477, CUDC-907, AEZS-136 or LY294002.

Exemplary antibodies that may be used in combination with the subject ADCs and/or PARPi may include any anti-cancer antibody known in the art, including but not limited to milatuzumab (anti-CD74), epratuzumab (anti-CD22), veltuzumab (anti-CD20), rituxumab (anti-CD20), obinutuzumab (anti-CD20), clivatuzumab (anti-MUC5ac), labretuzumab (anti-CEACAM5), L243 (anti-HLA-DR), alemtuzumab (anti-CD52), bevacizumab (anti-VEGF), cetuximab (anti-EGFR), gemtuzumab (anti-CD33), ibritumomab (anti-CD20), panitumumab (anti-EGFR), tositumomab (anti-CD20), magrolimab (anti-CD47) and trastuzumab (anti-HER2). Such known anti-cancer antibodies may be used in unconjugated (naked) form, or alternatively conjugated to at least one therapeutic agent, such as a DDR inhibitor or chemotherapeutic agent.

Another example of antibodies of use in combination therapies consists of checkpoint inhibitor antibodies. A variety of checkpoint inhibitor antibodies are known and any such known checkpoint inhibitor may be used in the subject combination therapy. Checkpoint inhibitor antibodies may bind to cytotoxic T-lymphocyte antigen 4 (CTLA4), programmed cell death protein 1 (PD-1) or programmed cell death 1 ligand 1 (PD-L1), as well as to other target proteins known to mediate checkpoint inhibition. Exemplary anti-PD-1 antibodies include pembrolizumab, nivolumab and pidilizumab. Exemplary anti-PD-L1 antibodies include MDX-1105, durvalumab, atezolizumab, MPDL3280A, and BMS-936559. Exemplary anti-CTLA4 antibodies include ipilimumab and tremelimumab. These and other known checkpoint inhibitors may be used in combination with an anti-Trop-2 ADC and/or PARPi.

In other alternatives, a DDR inhibitor besides PARPi may be used in combination with a subject ADC and/or a PARP inhibitor. Many such DDR inhibitors are known in the art and any such known DDR inhibitor may be used in the disclosed combinations. Inhibitors may be targeted against CDK12, RAD51, ATM, ATR, CHK1, CHK2, WEE1, or other known DDR proteins, as discussed above. CDK12 inhibitors may include dinaciclib, flavopiridol, roscovitine, THZ1 or THZ531 (Bitler et al., 2017, Gynecol Oncol 147:695-704; Krajewska et al., 2019, Nature Commun 10:1757; Paculova & Kohoutek, 2017, Cell Div 12:7). Inhibitors of RAD51 may include B02 ((E)-3-benzyl-2(2-(pyridin-3-yl)vinyl) quinazolin-4(3H)-one) (Huang & Mazin, 2014, PLoS ONE 9(6): e100993); RI-1 (3-chloro-1-(3,4-dichlorophenyl)-4-(4-morpholinyl)-1H-pyrrole-2,5-dione) (Budke et al., 2012, Nucl Acids Res 40:7347-57); DIDS (4,4′-diisothiocyanostilbene-2,2′-disulfonic acid) (Ishida et al., 2009, Nucl Acids Res 37:3367-76); halenaquinone (Takaku et al., 2011, Genes Cells 16:427-36); CYT-0851 (Cyteir Therapeutics, Inc.), IBR2 (Ferguson et al., 2018, J Pharm Exp Ther 364:46-54) or imatinib (Choudhury et al., 2009, Mol Cancer Ther 8:203-13). Inhibitors of ATM may include Wortmannin, CP-466722, KU-55933, KU-60019, KU-59403, AZD0156 CGK733, NVP-BEZ 235, Torin-2, fluoroquinoline 2 or SJ573017 (Weber & Ryan, 2015, Pharmacol Ther 149:124-38; Cruz et al., 2018, Ann Oncol 29:1203-10; Ronco et al., 2017, Med Chem Commun 8:295-319). Inhibitors of ATR may include NU6027, dactolisib, ETP46464, Torin 2, VE-821, berzosertib, AZ20, ceralasertib, M4344, EPT-46464, BAY1895344, AZD6738, CGK 733 or VX-970 (M6620). Inhibitors of CHK1 may include XL9844, UCN-01, CHIR-124, AZD7762, AZD1775, XL844, LY2603618, LY2606368 (prexasertib), GDC-0425, PD-321852, PF-477736, CBP501, CCT-244747, CEP-3891, SAR-020106, Arry-575, SRA737, V158411 or SCH 900776 (MK-8776). (See Wagner and Kaufmann, 2010, Pharmaceuticals 3:1311-34; Thompson and Eastman, 2013, Br J Clin Pharmacol 76:3; Ronco et al., 2017, Med Chem Commun 8:295-319). Inhibitors of CHK2 may include NSC205171, PV1019, CI2, CI3 (Gokare et al., 2016, Oncotarget 7:29520-30), 2-arylbenzimidazole, NSC109555, VRX0466617 or CCT241533 (Ronco et al., 2017, Med Chem Commun 8:295-319). Other DDR inhibitors include the WEE1 inhibitor AZD1775 (MK1775), the MRE11 inhibitor mirin, the BLM inhibitor M1216, the WRN inhibitor NSC19630, and the DNA-PKcs inhibitors Wortmannin, LY294002, MSC2490484A (M3814), VX-984 (M9831) and NU7026, (Srivastava & Raghavan, 2015, Chem Biol 22:17-29). These and other known DDR inhibitors may be used in combination therapy with an anti-Trop-2 ADC and/or PARPi.

Yet another class of therapeutic agent may comprise one or more immunomodulators. Immunomodulators of use may be selected from a cytokine, a stem cell growth factor, a lymphotoxin, an hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β, -γ or -λ, and stem cell growth factor, such as that designated “S1 factor.” Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -ß; mullerian-inhibiting substance; mouse gonadotropin-associated peptide; inhibin; activin; vascular endothelial growth factor; integrin; thrombopoietin (TPO); nerve growth factors such as NGF-ß; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin, tumor necrosis factor and lymphotoxin (LT). As used herein, the term cytokine includes proteins from natural sources or from recombinant cell culture and biologically active equivalents of the native sequence cytokines.

Chemokines of use include RANTES, MCAF, MIP1-alpha, MIP1-Beta and IP-10.

The person of ordinary skill will realize that the subject ADC and/or PARPi may be used alone or in combination with one or more other therapeutic agents, such as a second antibody, second antibody fragment, second ADC, radionuclide, toxin, drug, chemotherapeutic agent, radiation therapy, chemokine, cytokine, immunomodulator, enzyme, hormone, oligonucleotide, RNAi or siRNA. Such other agents may be used simultaneously or sequentially with the ADC and/or PARPi of the subject combination therapy.

Formulation and Administration

Suitable routes of administration of the ADC, PARPi and/or other therapeutic agents include, without limitation, oral, parenteral, subcutaneous, rectal, transmucosal, intestinal administration, intramuscular, intramedullary, intrathecal, direct intraventricular, intravenous, intravitreal, intraperitoneal, intranasal, or intraocular injections. In the case of the PARPi, the preferred route of administration is oral, preferably once a day. In the case of the ADC, the preferred route of administration is intravenous.

ADCs can be formulated according to known methods to prepare pharmaceutically useful compositions, whereby the ADC is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

In a preferred embodiment, the ADC is formulated in Good's biological buffer (pH 6-7), using a buffer selected from the group consisting of N-(2-acetamido)-2-aminoethanesulfonic acid (ACES); N-(2-acetamido)iminodiacetic acid (ADA); N,N-bis(2-hydroxyethyl)-2-aminoethanesulfonic acid (BES); 4-(2-hydroxyethyl)piperazine-1-ethanesulfonic acid (HEPES); 2-(N-morpholino)ethanesulfonic acid (MES); 3-(N-morpholino)propanesulfonic acid (MOPS); 3-(N-morpholinyl)-2-hydroxypropanesulfonic acid (MOPSO); and piperazine-N,N′-bis(2-ethanesulfonic acid) (Pipes). More preferred buffers are MES or MOPS, preferably in the concentration range of 20 to 100 mM, more preferably about 25 mM. Most preferred is 25 mM MES, pH 6.5. The formulation may further comprise 25 mM trehalose and 0.01% v/v polysorbate 80 as excipients, with the final buffer concentration modified to 22.25 mM as a result of added excipients. The preferred method of storage of ADC is as a lyophilized formulation, stored in the temperature range of −20° C. to 2° C., with the most preferred storage at 2° C. to 8° C.

The ADC can be formulated for intravenous administration via, for example, bolus injection, slow infusion or continuous infusion. Preferably, the ADC is infused over a period of less than about 4 hours, and more preferably, over a period of less than about 3 hours. For example, the first 25-50 mg could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

Generally, the dosage of an administered ADC for humans will vary depending upon such factors as the patient's age, weight, height, sex, general medical condition and previous medical history. It may be desirable to provide the recipient with a dosage of ADC that is in the range of from about 1 mg/kg to 24 mg/kg as a single intravenous infusion. A dosage of 1-20 mg/kg for a 70 kg patient, for example, is 70-1,400 mg, or 41-824 mg/m² for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy. Preferred dosages may include, but are not limited to, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, and 18 mg/kg. The dosage is preferably administered multiple times, once or twice a week, or as infrequently as once every 2, 3 or 4 weeks. A minimum dosage schedule of 4 weeks, more preferably 8 weeks, more preferably 16 weeks or longer may be used. The schedule of administration may comprise administration once or twice a week, on a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five week off; (vi) four weeks of therapy followed by one, two, three, four or five week off; (vii) five weeks of therapy followed by one, two, three, four or five week off; (viii) monthly and (ix) every 3 weeks. The cycle may be repeated 2, 4, 6, 8, 10, 12, 16 or 20 times or more.

In preferred embodiments, the ADC and PARPi are of use for therapy of cancer. Examples of cancers include, but are not limited to, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, glioblastomas, lung cancer including small-cell lung cancer, non-small cell lung cancer, adenocarcinoma of the lung and squamous carcinoma of the lung, cancer of the peritoneum, gastric or stomach cancer including gastrointestinal cancer, pancreatic cancer, glioblastoma multiforme, cervical cancer, ovarian cancer, liver cancer, bladder cancer, hepatoma, hepatocellular carcinoma, neuroendocrine tumors, medullary thyroid cancer, differentiated thyroid carcinoma, breast cancer, ovarian cancer, colon cancer, rectal cancer, endometrial cancer or uterine carcinoma, salivary gland carcinoma, kidney or renal cancer, prostate cancer, vulvar cancer, anal carcinoma, penile carcinoma, as well as head-and-neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Polycythemia vera, Parathyroid Cancer, Penile Cancer, Pheochromocytoma, Pituitary Tumor, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenström's macroglobulinemia, Wilms' tumor, and any other hyperproliferative disease, besides neoplasia, located in an organ system listed above. The person of ordinary skill will realize that different antibodies may be selected to treat different forms of cancer, and an antibody that binds to a target antigen that is expressed in the cancer to be treated will be selected for producing the subject ADC. In preferred embodiments, an anti-Trop-2 antibody is used to make an anti-Trop-2 ADC of use to treat cancers that express Trop-2.

The methods and compositions described and claimed herein may be used to treat malignant or premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)).

In preferred embodiments, the methods provided herein are used to inhibit growth, progression, and/or metastasis of cancers, in particular those listed above.

Any of the methods disclosed herein can be used in the manufacture of a medicament.

For example, provided herein is the use of an antibody-drug conjugate (ADC) that binds to Trop-2 in the manufacture of a medicament for treatment of cancer, the treatment comprising: administering to a human subject with a cancer that expresses Trop-2 an ADC that binds to Trop-2, wherein the drug component of the ADC is a topoisomerase inhibitor; and administering to the subject a Poly(ADP-ribose) polymerase inhibitor (PARPi), wherein the ADC and the PARPi are administered using a staggered dosing schedule.

In another example, provided herein is the use of sacitizumab govitecan in the manufacture of a medicament for treatment of metastatic TNBC cancer, the treatment comprising administering sacituzumab govitecan and talazoparib to a human metastatic TNBC patient on a staggered dosing schedule, wherein sacituzumab govitecan is administered on days 1 and 8 of a 21 day cycle at a dosage of 8 mg/kg to 10 mg/kg, and talazoparib is administered on days 15 to 21 of the 21 day cycle at a dosage of 0.5 mg/kg to 1.0 mg/kg.

Kits

Various embodiments may concern kits containing components suitable for treating diseased tissue in a patient. Exemplary kits may contain at least one ADC as described herein. A kit may also include a PARP inhibitor and/or other therapeutic agents. If the composition containing components for administration is not formulated for delivery via the alimentary canal, such as by oral delivery, a device capable of delivering the kit components through some other route may be included. One type of device, for applications such as parenteral delivery, is a syringe that is used to inject the composition into the body of a subject.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. In some embodiments, a sterile saline solution may be used for reconstitution. Kit components may be packaged and maintained sterilely within the containers. Another component that can be included is instructions to a person using a kit for its use.

### EXAMPLES

Various embodiments of the present disclosure are illustrated by the following examples, without limiting the scope thereof.

### Example 1. Scheduling of Combination Therapy with SG and Talazoparib PARPi in TNBC

### Background

Sacituzumab Govitecan (SG), the first antibody-drug conjugate approved for pretreated metastatic TNBC (mTNBC), is comprised of SN-38 (active metabolite of irinotecan), a topoisomerase I (TOP1) inhibitor, coupled via a hydrolyzable linker to monoclonal antibody targeting trophoblast cell surface antigen 2 (Trop-2), an epithelial antigen overexpressed in various solid tumors, including mTNBC. While SG monotherapy has demonstrated a survival benefit in 2L+mTNBC, further improvement is needed for patients with mTNBC. Poly (ADP-ribose) polymerase inhibitors (PARPi) prevent PARP-dependent replication fork reversal and block resolution of TOP1 cleavage complexes (TOP1CCs) induced by TOP1 inhibitors, thus unmasking the inability of remaining pathways including Homologous Recombinant to repair DNA damage. However, previous clinical trials combining PARPi with standard TOP1 inhibitors (irinotecan, topetecan) were terminated early due to dose-limiting myelosuppression. To address the unmet need, we evaluated the combination of SG with a PARP inhibitor in both pre-clinical models and phase 1b clinical trial.

TNBC is a biologically aggressive form of breast cancer defined as the absence of estrogen and progesterone receptors and lack of human epidermal growth factor receptor 2 (HER2) gene amplification. It is associated with a high mortality rate with a median survival of 10-13 months from the time of metastasis (Aditya Bardia et al. (2021) The New England Journal of Medicine 384 (16): 1529-41). Sacituzumab govitecan (SG), an antibody-drug conjugate targeting TROP2 with the TOP1 inhibitor SN-38 payload, has demonstrated higher clinical activity than standard chemotherapy for patients with pre-treated metastatic triple negative breast cancer (Goldenberg et al. (2015) Oncotarget 6 (26): 22496-512; Aditya Bardia et al. (2019) The New England Journal of Medicine 380 (8): 741-51; A. Bardia et al. (2021) The New England Journal of Medicine 384 (16): 1529-41; Aditya Bardia et al. (2017) Journal of Clinical Oncology: Official Journal of the American Society of Clinical Oncology 35 (19): 2141-48). In the phase III ASCENT study, SG was compared with treatment of physician's choice (eribulin, vinorelbine, capecitabine, or gemcitabine) in patients with previously treated mTNBC. The median progression-free survival was 5.6 months with SG vs 1.7 months with standard chemotherapy and the median overall survival was 12.1 months with SG vs 6.7 months with standard chemotherapy (Aditya Bardia et al. (2022) Journal of Clinical Orthodontics: JCO 40 (16_suppl): 1071-1071). While use of SG as a monotherapy has been a substantial advancement in treatment options for metastatic breast cancer, there is a significant unmet clinical need for novel combinatorial strategies to further improve outcomes for patients with mTNBC.

TNBC frequently displays high genomic instability, making it more sensitive to DNA-damaging agents and DNA-repair inhibitors such as PARP inhibitors (Guo and Wang (2021) Frontiers in Cell and Developmental Biology 9 (July): 701073; Kwei et al. (2010) Molecular Oncology 4 (3): 255-66; Bianchini et al. (2016) Nature Reviews. Clinical Oncology 13 (11): 674-90). Combination therapy of SG with PARP inhibitors is of great interest given the complementary mechanisms of action, lack of cross-resistance, and therapeutic synergy demonstrated in multiple in vivo and in vitro models (Cardillo et al. (2017) Clinical Cancer Research: An Official Journal of the American Association for Cancer Research 23 (13): 3405-15). It has been shown that agents that inhibit TOP1 and therefore damage DNA synergize with PARPi to deter the growth of many human tumor cell lines including those of breast cancer (Kummar et al. (2011) Cancer Research 71 (17): 5626-34; LoRusso et al. (2016) Clinical Cancer Research: An Official Journal of the American Association for Cancer Research 22 (13): 3227-37; Znojek, Willmore, and Curtin (2014) British Journal of Cancer 111 (7):1319-26; Smith et al. (2005) Clinical Cancer Research: An Official Journal of the American Association for Cancer Research 11 (23): 8449-57).

In pre-clinical models, the SG and PARPi combination increased dsDNA breaks in TROP2-expressing cells (>5-fold increase in pH2A.X levels), likely resulting from combined effects of stabilized TOP1CC and inhibited repair of TOP1i-induced double-strand breaks (B. B. Das et al. (2014) Nucleic Acids Research 42 (7): 4435-49; S. K. Das et al. (2016) Nucleic Acids Research 44 (17): 8363-75; Cardillo et al. (2017) Clinical Cancer Research: An Official Journal of the American Association for Cancer Research 23 (13): 3405-15), and consequently resulted in apoptosis (synthetic lethality). In xenograft models, the combination of SG and PARPi resulted in significantly higher antitumor activity than was observed with monotherapy, including partial responses in all mice, complete responses in 30%, and significantly delayed time to progression compared to monotherapy (p<0.0017) (Cardillo et al. 2017). However, prior clinical trials that used combination intravenous TOP1 inhibitors with PARP inhibitors resulted in high levels of toxicity (Kummar et al. 2011). Dose-limiting myelosuppression severely limited the ability to dose escalate both PARP inhibitor and traditional chemotherapy in several clinical studies, precluding this combination from moving beyond early phase studies (Thomas and Pommier (2019) Clinical Cancer Research: An Official Journal of the American Association for Cancer Research 25 (22): 6581-89).

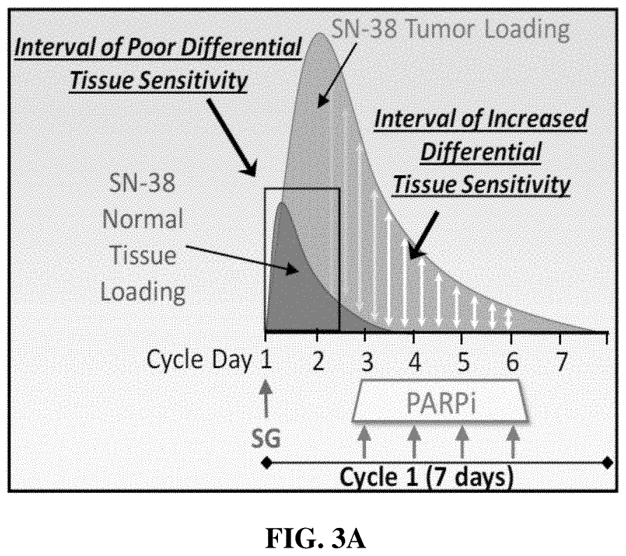

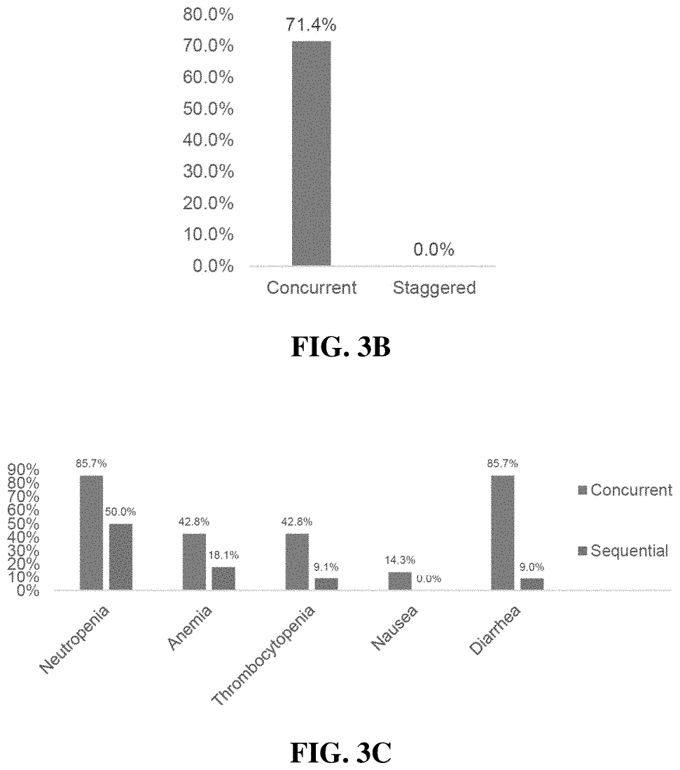

We hypothesized i) that the antibody-based delivery mechanism would provide a more favorable therapeutic window for the combination, and ii) that increased ratio of tumor-to-normal cell SN-38 delivery by SG would provide a temporal window to allow sequential dosing (SG followed by PARPi) to achieve potent tumor DNA damage and cell killing while further protecting normal cells (FIG. 3A). To test these hypotheses, we conducted an investigator-initiated multi-center study to evaluate the safety/tolerability and efficacy of SG and talazoparib combination therapy for patients with metastatic TNBC. As expected, the concurrent dosing of SG and talazoparib was associated with significant toxicity. We then evaluated a novel sequential strategy with SG and talazoparib, supported by mechanistic studies. Here we present the results from the clinical trial and complementary pre-clinical models

Overview:

First, a multi-center study was conducted to evaluate the safety/tolerability and efficacy of SG and talazoparib combination therapy for patients with metastatic TNBC. The concurrent dosing of SG and talazoparib was found to be associated with significant toxicity. Next, a clinical study was conducted using a staggered dosing schedule with SG and talazoparib, supported by mechanistic studies.

Materials and Methods:

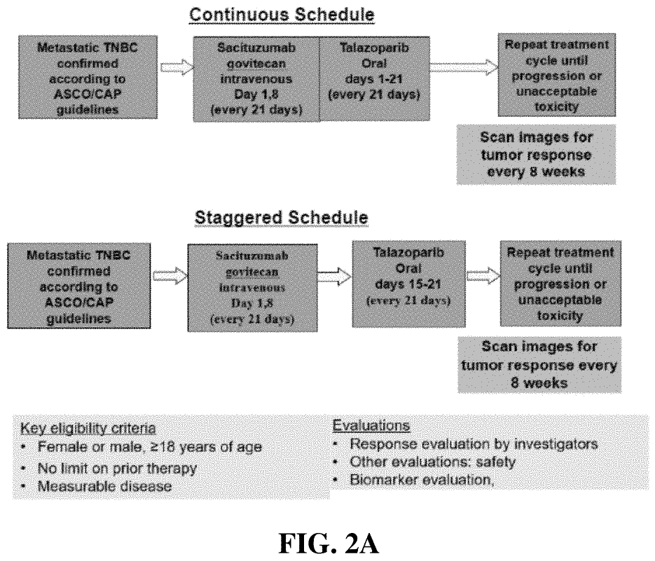

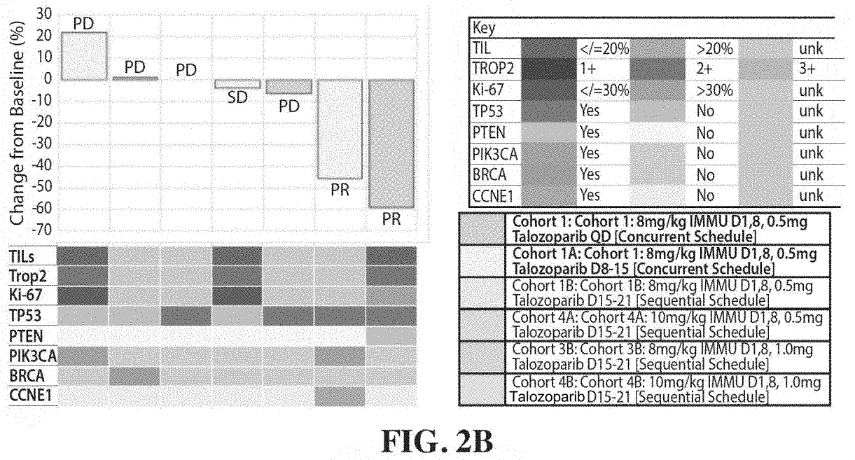

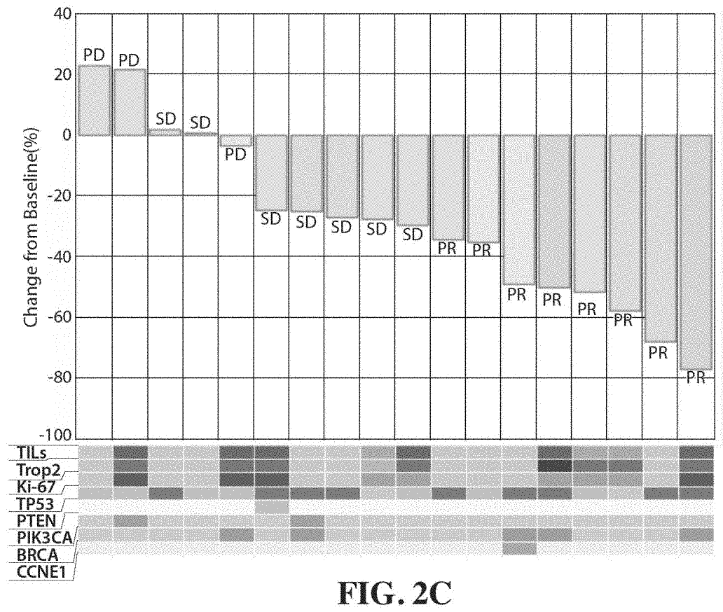

Study Design: In a Phase 1b/2, open-label study, SG was administered in combination with talazoparib for patients with mTNBC (clinicaltrials.gov#NCT04039230; FIG. 2A). Treatment cycles were continued until unacceptable toxicity or progression of disease at the discretion of the treating physician. Within 4 weeks prior to the first dose of study treatment administration, baseline evaluations were completed which included patient medical and surgical history, a physical examination with vital signs and performance evaluation, laboratories, and tumor assessment imaging. Once on active treatment, patients were administered SG and talazoparib over a 21 day cycle or 28 day cycle. The cycles were continued in the absence of unacceptable toxicity or progression of disease. During treatment, study procedures include physical examinations, vital signs, blood labs, serum samples, concomitant medications, adverse events, EKG, and restaging scans. All patients were monitored closely over the course of their treatment and NCI CTC v5.0 was used to grade all adverse events and provide dose reduction, delay, or cessation guidelines in the event of treatment-related toxicity. The standard 3+3 dose-escalation design was used in this study, with additional patients allowed to enroll for research purposes at the discretion of the principal investigator.

Study Population: Patients enrolled were 18 years of age or older with histological or cytological confirmation of TNBC as determined by the local institution, with metastatic disease documented by CT or MRI imaging, and currently have measurable disease by CT/MRI. All patients had an ECOG performance score of <1 at screening, and adequate bone marrow, hepatic, and renal function. Patients were at least 2 weeks beyond prior anti-cancer treatment.

Study Medications: SG was administered intravenously day 1, 8 every 21 days on an outpatient basis. Talazoparib was provided as capsules for oral administration. When on active treatment, other anti-cancer treatment was not permitted during this study. Palliative and/or supportive medications and procedures were permitted at the physician's discretion.

Endpoints: Safety and tolerability of SG in combination with talazoparib was evaluated from adverse events, standard safety laboratories, physical examination, vital signs, and EKG. All adverse events and abnormal laboratories were classified for severity using NCI CTCAE v5.0 toxicity grades. Treatment efficacy was evaluated from CT or MRI scans using RECIST 1.1. criteria to classify tumor response, time to onset of objective response, duration of objective response, progression-free survival (PFS) and overall survival (OS) was documented. After discontinuation of study treatment, all patients were followed every 8 weeks for survival follow-up. Follow-up visits could be in-clinic or by telephone and were meant to document any further therapy administered for the patient's breast cancer. Adverse event reporting continued for 30 days after the last dose of study treatment.

Cell Lines and Cell Culture: All cell lines were obtained from the MGH Center for Molecular Therapeutics cell bank in and underwent high-density SNP typing to confirm their identity. All experiments shown were performed within less than 6 months' passage of all lines since acquisition. Cells were maintained at 37° C. in 5% CO2. MDA-MB-468 cells were grown in RPMI (Lonza) supplemented with 10% FBS (SAFC), 1% penicillin (Gibco), streptomycin (Gibco). WI-38 cells were grown in DMEM (Lonza) supplemented with 10% FBS (SAFC), 1% penicillin (Gibco), streptomycin (Gibco).

Western Blotting: Cells cultured in 10 cm dish were collected with 500 μL fractionation buffer (250 mM sucrose, 20 mM HEPES (pH 7.4), 10 mM KCl, 2 mM MgCl2, 1 mM EDTA, 1 mA EGTA, 1 mM DTT, proteinase inhibitor cocktail) and passed through a 25-gauge needle 10 times. The cell suspension was centrifuged at 720 g for 5 min, and the supernatant was then centrifuged at 10,000 g for 5 min. The supernatant was then centrifuged at 100,000 g for 1 h. The pellet was washed by adding 500 ul of fractionation buffer and the pellet was resuspended by pipetting and passing through a 25G needle. The sample was centrifuged for 1 h. The membrane pellet was resuspended with SDS sample buffer. Protein samples were mixed with SDS sample buffer and boiled for 10 minutes before being subjected to SDS-PAGE. The protein samples on the SDS-PAGE gel were then transferred onto PVDF membrane (Millipore), which was blocked by 5% nonfat milk in PBST (PBS plus 0.02% Tween 20) at room temperature for 1 hour. Then, the PVDF membrane was incubated with primary antibodies diluted in 3% BSA in PBST at 4° C. overnight and horseradish peroxidase-conjugated secondary antibodies (Sigma) diluted in 3% BSA in PBST at room temperature for 2 hours. The signal was detected by enhanced chemiluminescence solution (PerkinElmer)

Modification of the RADAR assay for detection of TOP1CC: Detection of TOP1CC was performed according to the published protocol (PMID: 34408146) with minor modification. After SG and/or TZP treatment, 1×10⁶ cells per condition were washed with 1×PBS and lysed with 600 μl DNAzol (Invitrogen) at 4° C. for 10 min, followed by precipitation with 300 μl 100% ethanol. The nucleic acids were collected by centrifugation at 15,000 rpm for 10 min at 4° C., washed with 75% ethanol twice and resuspended in 200 μl TE buffer. The samples were then heated at 65° C. for 15 min, followed by shearing with sonication (40% output for 10 s pulse and 10 s rest for four times). The samples were centrifuged at 15,000 rpm for 5 min at 4° C. and the supernatant was collected and treated with 100 μg/ml RNase A (Thermo Fisher Scientific) for 1 h at 4° C., followed by the addition of 1/10 volume of 3 M sodium acetate (PH 5.5) and 2.5 volume of 100% ethanol. After 20 min of centrifugation at 15,000 rpm at 4° C., the DNA pellet was resuspended in 100 μl TE buffer. The concentration of the sample was quantified by NanoDrop. 10 μg of DNA from each condition were digested with 50 units of micrococcal nuclease (Thermo Fisher Scientific) in presence of 5 mM CaCl2 at 37° C. for 30 min. The samples were then mixed with 5×SDS sample buffer and subject to the Western Blotting by using anti-TOP1 antibody (Abcam, #ab109374). 2 μg of dsDNA for each condition was subjected to dot-blot by using Nylone membrane (Santa Cruz) for immunoblotting with anti-dsDNA antibody (abcam, #ab27156) as a loading control to verify that amounts of DNA were digested with micrococcal nuclease.

Flow Cytometry: For flow cytometry detection of γH2AX (also known as g-H2AX), 1×10⁶ cells were collected and fixed with 4% paraformaldehyde at 4° C. for 20 min and then permeabilized with 0.25% Triton X-100 in PBS. After blocking with 2% BSA in PBS at 4° C. for 20 min, the cells were stained with Alexa Fluor® 488 conjugated anti-phospho Histone H2A.X antibody (EMD Millipore, clone JBW301, #05-636-AF488) at 4° C. for 1 h. The cells were then washed twice with 2% BSA in PBS and counterstained with DAPI (Abcam, #ab228549). For flow cytometry detection of apoptotic cells, 1×10⁶ cells were collected, and the staining was performed by using eBioscience™ Annexin V Apoptosis Detection Kit APC (Invitrogen, #88-8007) according to manufacturer's instruction. Samples were examined using a FACSAria flow cytometer. Analysis was conducted with FlowJo using Cell Quest software (Becton Dickinson, Franklin Lakes, NJ).

Immunofluorescence: To visualize TOP1CC by immunofluorescence, cells were plated into 96-well plates having #1.5 glass coverslips (Cellvis, P98-1.5H-N). Cells were then treated with SG and/or TZP, washed with 1×PBS, and fixed with 4% paraformaldehyde (PFA) at 37° C. for 10 min. Thereafter, cells were washed three times with 1×PBS and permeabilized with 0.1% NaCitrate/0.1% Triton X-100 in ddH2O at room temperature for 5 min. Cells were then treated with 0.5% SDS in PBS at room temperature for 5 min and washed five times with 0.25% BSA/0.1% Tween-20 in PBS. After blocking in 2% BSA/10% normal goat serum/0.1% Tween-20 in PBS at room temperature for 30 min, cells were incubated with anti-TOP1CC antibody (EMD Millipore, MABE1084) diluted 1:100 in blocking buffer at room temperature for 1 h. Following incubation with an Alexa Fluor® 488 secondary antibody (Jackson ImmunoResearch) at room temperature for 30 min and DAPI counterstaining, cells were imaged using a Nikon A1R confocal microscope with a 60× oil immersion objective. Images were compiled and quantified using ImageJ software.

Results:

Baseline characteristics were balanced between two cohorts: Between October 2019 and April 2021, 29 patients were enrolled in the clinical trial. Initially, 7 patients were enrolled in the concurrent cohort (October 2019-December 2019), but subsequent enrollment in the concurrent cohort was discontinued due to significant toxicity. Further enrollment was in the sequential cohort and 22 patients were enrolled between January 2020 and April 2021. There were no significant differences in baseline characteristics between the two cohorts, including median age, race, ECOG PS, prior lines of therapy, and site of metastasis (Table 1).

TABLE 1
Baseline Characteristics of the study population

| | Continuous | | Sequential | | p = |
|---|---|---|---|---|---|
| Characteristic | n | Percentage | n | Percentage | value |
| Age (median) | 47.1 | | 54.4 | | 0.54 |
| Age 50 | | | | | 0.2 |
| </=50 | 4 | 57% | 6 | 30% | |
| >50 | 3 | 43% | 14 | 70% | |
| Sex | | | | | |
| Female | 7 | 100% | 20 | 100% | |
| Race | | | | | 0.3 |
| Asian | 2 | 29% | 1 | 5% | |
| Black or African American | 0 | 0% | 1 | 5% | |
| More than One Race | 0 | 0% | 2 | 10% | |
| White | 5 | 71% | 16 | 80% | |
| Ethnicity | | | | | 0.55 |
| Ethnicity Not Known | 0 | 0% | 1 | 1% | |
| Non-Hispanic | 7 | 100% | 19 | 95% | |
| ECOG PS | | | | | 0.74 |
| 0 | 3 | 43% | 10 | 50% | |
| 1 | 4 | 57% | 10 | 50% | |
| Time from Metastatic Diagnosis to | 10.3 | | 7.3 | | 0.35 |
| Enrollment (median) | | | | | |
| Previous Total Treatment Lines (median) | 6 | | 4 | | 0.17 |
| Previous Chemotherapy Drugs | | | | | NS |
| Taxanes | 4 | 57% | 20 | 91% | |
| Anthracyclines | 6 | 86% | 18 | 82% | |
| Cyclophosphamides | 6 | 86% | 17 | 77% | |
| Carboplatin | 4 | 57% | 10 | 45% | |
| Capecitabine | 5 | 71% | 11 | 50% | |
| Sacituzumab Govitecan | 0 | 0% | 1 | 5% | |
| Previous Immunotherapy | 0 | 0% | 2 | 9% | NS |
| Previous Parp Inhibitor | 1 | 14% | 5 | 23% | NS |
| Previous PD-1 or PDL-1 Inhibitor | 1 | 14% | 4 | 18% | NS |

*NS = Not-significant

Lower toxicity was noted with staggered vs. concurrent schedule: The concurrent dosing schedule was associated with significant toxicity, with 5/7 (71.4%) of patients experiencing DLTs, including febrile neutropenia. In contrast, no patient (0%) experienced DLT with the sequential schedule (FIG. 3B). Similarly, the incidence of adverse effects including neutropenia, anemia, thrombocytopenia, diarrhea was higher in the concurrent cohort vs the sequential cohort (FIG. 3C, Table 2).

TABLE 2
Adverse Effects

| | Continuous | | Staggered | |
|---|---|---|---|---|
| Toxicity | Grade ½ | Grade ¾ | Grade ½ | Grade ¾ |
| Neutropenia | 0 | 6 | 2 | 11 |
| Anemia | 2 | 3 | 9 | 4 |
| Thrombocytopenia | 1 | 3 | 8 | 2 |
| Diarrhea | 1 | 6 | 12 | 2 |
| Nausea | 4 | 1 | 12 | 0 |
| Rash | 2 | 0 | 3 | 0 |
| Infusion Reaction | 0 | 0 | 1 | 1 |

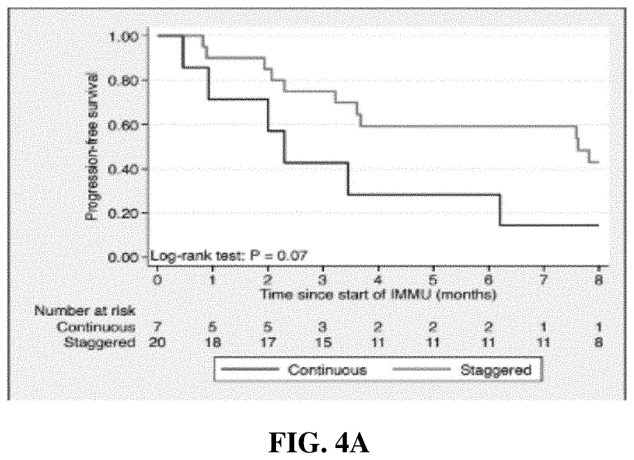

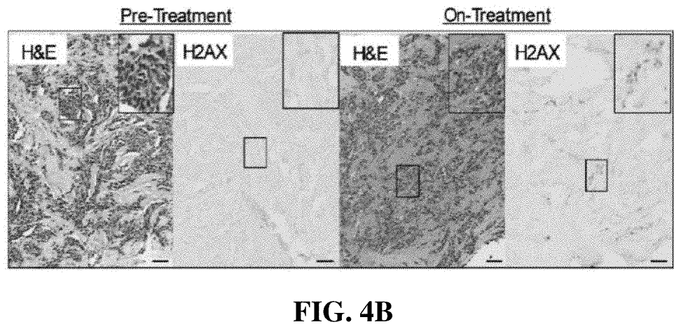

Higher efficacy was noted with staggered vs concurrent schedule: In terms of efficacy, only two patients in the concurrent schedule had a partial response (FIG. 2B). In contrast, eight patients in the sequential cohort had a confirmed partial response (FIG. 2C). Similarly, the median PFS was higher in patients enrolled in sequential cohort (7.6 months), as compared to those in concurrent cohort (2.3 months), as outlined in FIG. 4A. The overall survival was also longer in patients enrolled in the sequential cohort (11.1 months) as compared to those in concurrent cohort (4.3 months), and the clinical benefit rate was also higher in patients treated with sequential schedule than concurrent schedule (Table 3)

TABLE 3
Treatment Efficacy

| | Continuous | Sequential |
|---|---|---|
| Median progression-free survival (mo) | 2.3 | 7.62 |
| Median overall survival (mo) | 4.34 | 11.17 |
| Objective Response | 2 | 8 |
| Clinical Benefit | 3 | 15 |
| Stable Disease | 1 | 8 |
| Stable disease for >/= 6 months | 0 | 7 |
| Progressive Disease | 4 | 4 |
| Response could not be evaluated | 0 | 4 |

Biomarker results were consistent with the genomic spectrum of TNBC, including tumors with TP53 mutation, PIK3CA mutation, PTEN mutation (FIG. 2B, FIG. 2C). Two patients had known cyclin-E amplifications and had a partial response. Similarly, there was a trend towards higher objective response rate among tumors with higher TIL infiltration or proliferation index (FIG. 2B, FIG. 2C).

Pharmacodynamic inhibition was noted with staggered dosing: Pre-treatment and on-treatment biopsy (cycle 2 day 5) samples were collected from a patient treated with staggered dosing and stained for phosphorylated H2AX (g-H2AX) a canonical indicator of DNA double-strand breaks (DSBs) that is low/negative in most TNBC at baseline but is increased in both tumor and normal cells in the setting of persistent DNA damage. The biomarker analysis demonstrated absence of g-H2AX in the tumor at baseline but accumulation of g-H2AX post-treatment, consistent with therapy-induced DNA damage (FIG. 4B). Clinically, the patient had a prolonged therapeutic response to the combination with sequential dosing of SG and talazoparib.

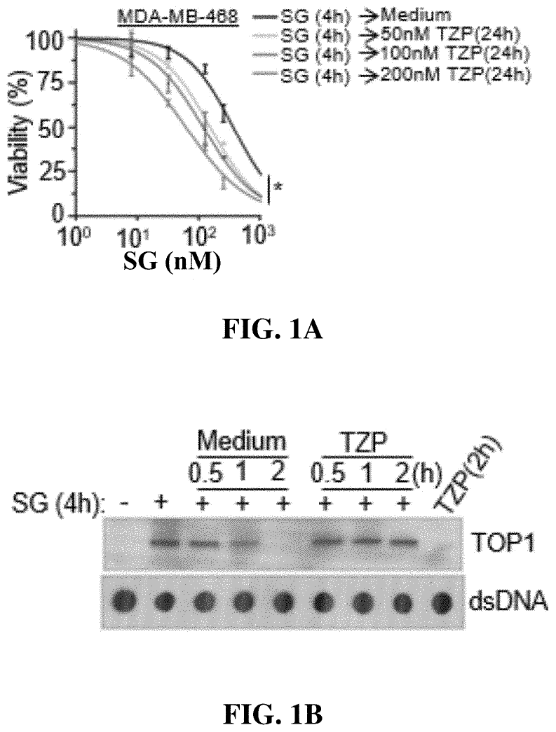

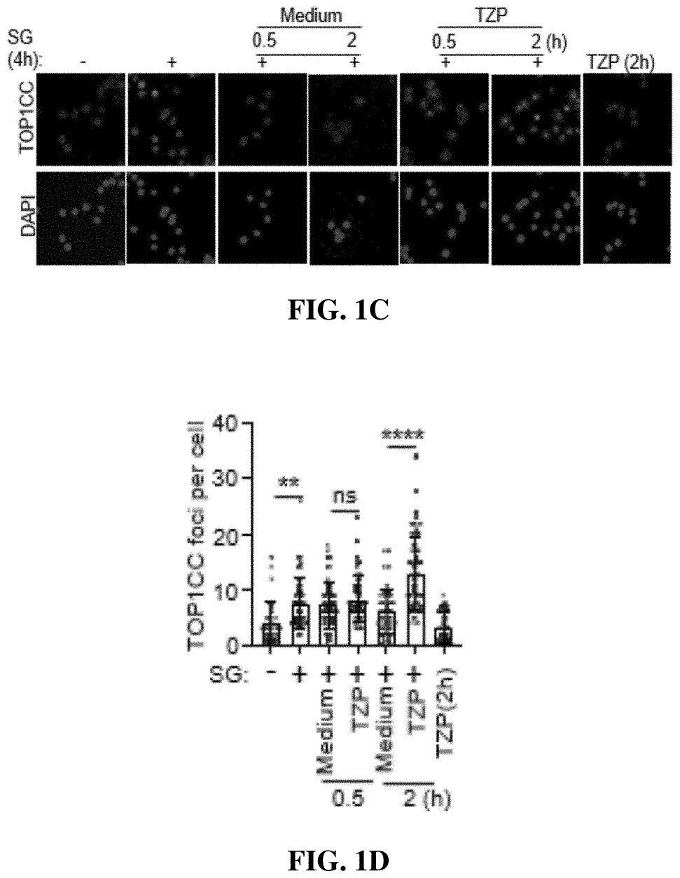

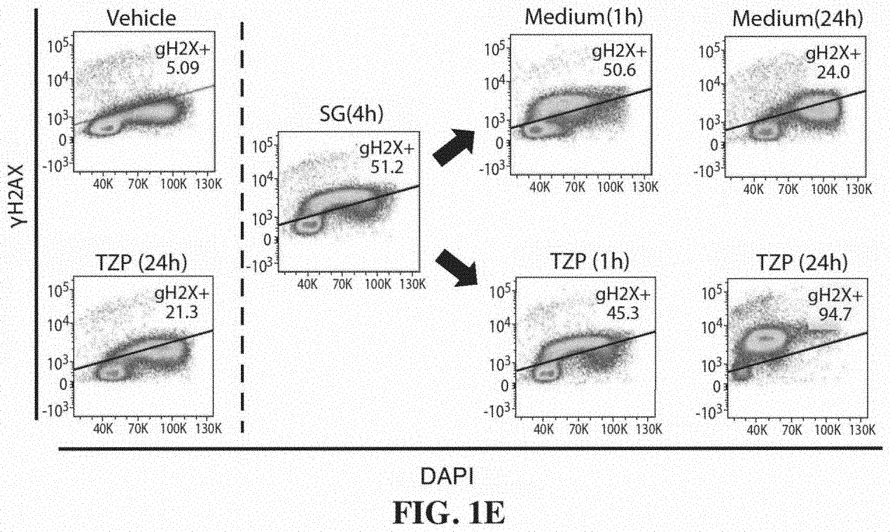

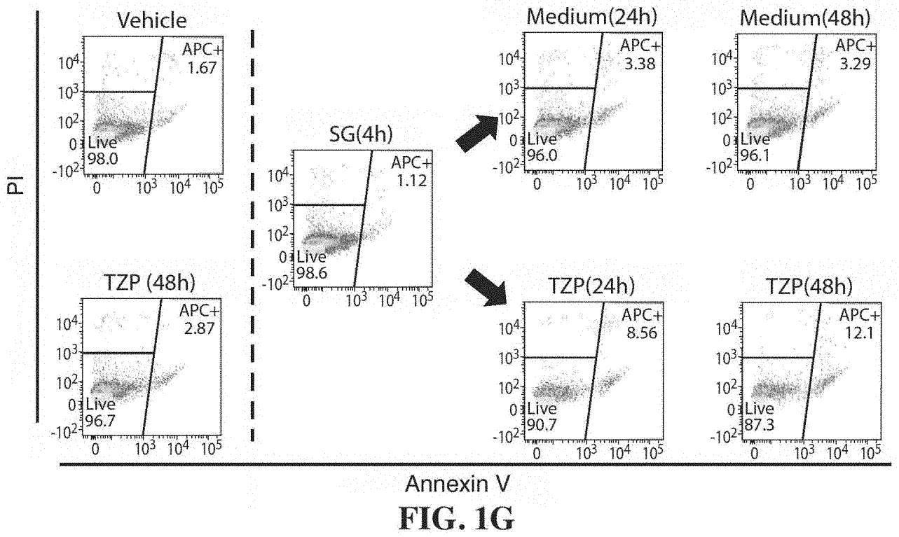

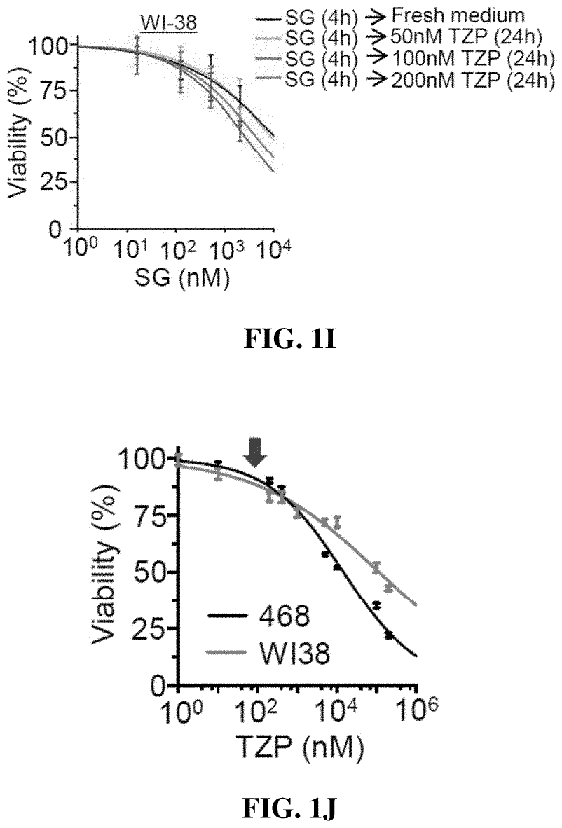

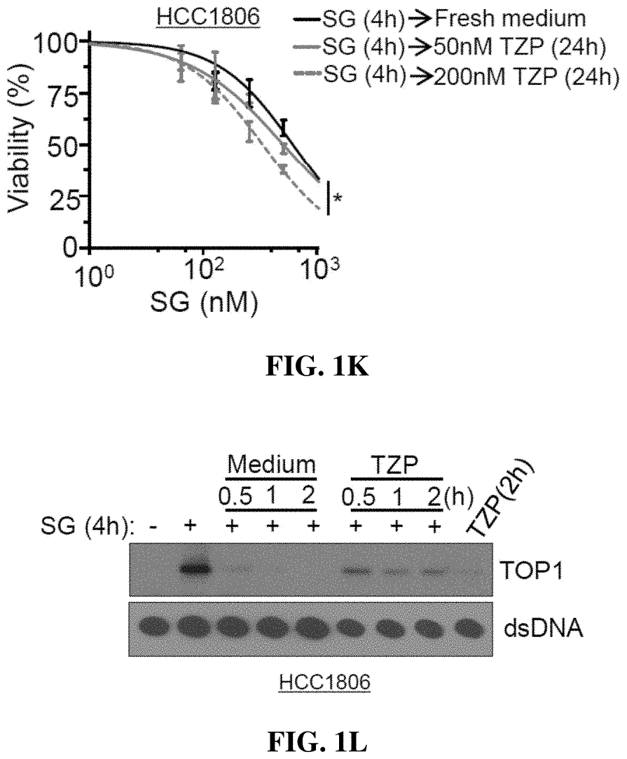

Synergistic cytotoxicity and TOP1CC stabilization was noted with staggered SG/PARPi: Cell-based TNBC models were used to quantify cytotoxicity and assess potential mechanisms of sequential SG followed by PARPi treatment. TROP2-expressing TNBC cells or normal diploid cells were exposed to SG, followed by washout, then PARPi treatment with Talazoparib. Despite the temporal separation, SG followed by PARPi (at a minimally toxic dose) showed a synergistic and dose-dependent enhancement of SG toxicity at clinically achievable doses, supporting the rationale for sequential (e.g., staggered) dosing (FIG. 1A, FIG. 1K). In contrast, normal diploid cells (WI-38) showed little SG sensitivity and no appreciable enhancement with the addition of PARPi (FIG. 1L).

Without wishing to be bound by any theory, sequential PARPi is believed to enhance SG toxicity through stabilization of TOP1CC that persist following SG washout due to the high delivered SN38 concentration in tumor cells (FIG. 3A). TOP1CC was quantified by two methods: the RADAR (Rapid Approach to DNA Adduct Recovery) assay, which detects TOP1 covalently bound to DNA, and by immunofluorescence. TOP1CC as assessed by either method were detectable immediately following SG washout but resolved entirely within two hours, whereas in the presence of sequential PARPi no resolution of TOP1CC was observed (FIG. 1B, FIG. 1C). Both dose-dependent enhancement of SG toxicity and stabilized TOP1CC with sequential PARPi were observed in multiple TNBC models (FIG. 1K, FIG. 1L). Persistent TOP1CC are expected to lead to DNA damage including double-strand DNA breaks, which were quantified using flow cytometry to assess phosphorylated histone H2AX (γH2AX) staining. In the absence of PARPi, washout of SG led to resolution of DNA damage within 24 hours, whereas in the presence of sequential PARPi following SG washout, cells continued to accumulate high levels of DNA damage (FIG. 1E, FIG. 1F). Correspondingly, apoptosis was consistently and significantly increased by the addition of PARPi post SG washout (FIG. 1G, FIG. 1H). Collectively, these findings support the rationale for sequential (e.g., staggered) SG/PARPi as a means to limit off-target toxicity, allowing improved tolerance while enhancing tumor cell killing through a mechanism involving stabilization of TOP1CC

Methods and Results By conducting a well-calibrated genome-wide CRISPR screen with SG in TNBC cells, the PARP pathway was first identified as the top resistance hit to SG, highlighting the role of PARPi to overcome SG resistance. Second, in pre-clinical models it was demonstrated that targeted antibody-based delivery of SN-38 increased the ratio of tumor-to-normal cell SN-38, resulting in stabilized TOP1CCs, enhanced DNA damage and increased cytotoxicity with the combination, selectively in tumor cells but not normal cells, despite temporal separation of SG and PARPi exposure. Moreover, a phase 1b clinical trial was conducted combining SG with PARPi (talazoparib) in patients with mTNBC (NCT04039230). Inclusion criteria included female patients ≥18 years of age with mTNBC (per ASCO/CAP guidelines), measurable disease, and previous treatment with at least one prior therapeutic regimen for mTNBC. Restaging scans obtained every 8 weeks and clinical outcomes were assessed by Objective Response Rate per RECIST v1.1.

In the phase 1b clinical trial (SG day 1,8 every 21 days with talazoparib), 32 patients were enrolled in the dose-escalation portion, including 25 patients treated with the staggered schedule. While concurrent administration of SG and talazoparib was associated with multiple dose-limiting toxicities (DLTs) due to severe myelosuppression (4 out of 7 patients had febrile neutropenia), the staggered schedule with supportive therapy was relatively well-tolerated without DLTs. Furthermore, the staggered schedule demonstrated promising clinical activity. Molecular analysis of paired pre-treatment and on-treatment specimens demonstrated γ-H2AX accumulation, confirming pharmacodynamic inhibition with combination therapy. The dose-escalation portion of the clinical trial successfully completed enrollment in April 2021 with a recommended phase-2 dose (R2PD) of sequential SG (10 mg/kg on days 1,8) with talazoparib (1 mg on days 15-21), every 21 days. Preclinical results are described in FIGS. 1A-1C. The clinical trial design is depicted in FIG. 2A. Efficacy results (RECIST) for the continuous dosing schedule are shown in FIG. 2B. Efficacy results (RECIST) for the staggered dosing schedule are shown in FIG. 2C.

### CONCLUSION

This Example demonstrates that that antibody-mediated tumor-selective delivery of a TOP1 inhibitor, such as SN38, via an anti-Trop-2 antibody-drug-conjugate (anti-Trop-2), such as sacituzumab govitecan (SG), can enable sequential dosing of SG with a PARP inhibitor (e.g., talazoparib) in a staggered dosing schedule to enhance the therapeutic window and allow delivery of the combination with less toxicity.

Specifically, this Example demonstrates that i) that an antibody-based delivery mechanism can provide a more favorable therapeutic window for an anti-Trop-2 ADC/PARPi combination, and ii) that increased ratio of tumor-to-normal cell TOP1 inhibitor (e.g., SN-38) delivery by an anti-Trop-2 ADC (e.g., SG) can provide a temporal window to allow sequential dosing (SG followed by PARPi) to achieve potent tumor DNA damage and cell killing while further protecting normal cells (FIG. 1A).

This Example further provides a mechanistic rationale and clinical proof-of-principle supporting sequential (e.g., staggered) dosing of SG and PARPi to minimize toxicity and maximize efficacy. The dose-escalation portion of clinical trial successfully completed enrollment with a recommended phase-2 dose (R2PD) of sequential SG (10 mg/kg on days 1,8) with talazoparib (1 mg on days 15-21) every 21 days. The study highlights a new paradigm for therapeutic combinations involving antibody-drug conjugates, whereby the high levels of payload drug delivered to tumor vs. normal cells offers opportunities for innovative scheduling of mechanism-based combinations that may overcome otherwise prohibitive toxicities for cancer patients including those with mTNBC.

Potential synergy between PARPi and TOP1i has been observed in previous studies, leading to clinical trials combining PARP inhibitors with standard TOP1 inhibitors. However, several such studies were terminated early due to unacceptable myelosuppression caused by the combination. For example, the PARP inhibitor veliparib in combination with topotecan was found to be highly myelosuppressive, requiring dose reductions for both agents with the MTD of veliparib and topotecan only 3% and 40% of the respective single-agent MTDs (Thomas and Pommier (2019) Clinical Cancer Research: An Official Journal of the American Association for Cancer Research 25 (22): 6581-89). Additionally, in a phase 2 clinical trial combining olaparib and irinotecan in colorectal cancer, continuous olaparib administration was associated with higher-than-expected toxicities and considered intolerable (Yarchoan M et al (2017) Oncotarget 8(27):44073-81). Thus, despite a strong mechanistic rationale for the combination of two active therapeutics, systemic delivery of PARPi and TOP1i is associated with an insufficient therapeutic window and proved unsuccessful due to severe toxicity.

This Example further showed that concurrent administration of SG and talazoparib produced unacceptable myelosuppression, which suggests that exploiting specificity of payload drug delivery by the ADC alone was not sufficient to make the combination tolerable. Nonetheless, the specificity and high intratumoral SN-38 delivery was shown to provide a temporal window, enabling a novel dosing strategy with sequential (e.g., staggered) treatment that indeed demonstrated improved tolerability and allowed the study to proceed.

The findings illustrated in this Example are believed to have broad implications for combination therapy with ADCs generally. Clinically, it was demonstrated that an antibody-based delivery mechanism can provide a more favorable therapeutic window for an otherwise toxic combination and increased delivery of a payload to cancer cells while sparing normal cells. This Example further demonstrated the existence of a temporal window, supporting sequential (e.g., staggered) rather than concurrent dosing that can improve the efficacy-toxicity ratio. Sequential delivery of a DNA damaging agent and a repair inhibitor can also be utilized for other ADCs, particularly those with TOP1i payloads such as trastuzumab deruxtecan, datapotamab deruxtecan, and patritumab deruxtecan, all of which are in advanced stages of clinical development in oncology. More broadly, the tumor-selective delivery of antibody-targeted drugs may facilitate combinations with diverse other agents with minimal toxicity, including the possibility of revisiting previously discarded options in combination with novel ADCs.

In summary, the sequential (e.g., staggered) dosing of SG and PARPi, leveraging the selective drug delivery mechanism of SG to minimize toxicity while maintaining efficacy, was clinically feasible and demonstrated encouraging evidence of clinical activity with objective responses among pre-treated patients with mTNBC. The translational study described here illustrates the potential of antibody drug conjugate-based therapy to facilitate novel, mechanism-based dosing strategies that render viable previously rejected chemotherapy combinations in oncology, e.g., for patients with mTNBC.

These results support the surprising and unexpected conclusion that the staggered dosing of a PARP inhibitor and a topoisomerase-I inhibiting ADC can be utilized to minimize adverse events associated with the combination therapy, while maintaining efficacy against targeted tumor tissues.

From the foregoing description, one skilled in the art can easily ascertain the essential characteristics of this disclosure, and without departing from the spirit and scope thereof, can make various changes and modifications of the disclosure to adapt it to various usage and conditions without undue experimentation. All patents, patent applications and publications cited herein are incorporated by reference.

1. A method of treating cancer, comprising:
a) administering to a human subject with a cancer that expresses Trop-2 an antibody-drug conjugate (ADC) that binds to Trop-2, wherein the drug component of the ADC is a topoisomerase inhibitor; and
b) administering to the subject a Poly(ADP-ribose) polymerase inhibitor (PARPi), wherein the ADC and the PARPi are administered using a staggered dosing schedule.

2. The method of claim 1, wherein:
(i) the topoisomerase inhibitor is an inhibitor of topoisomerase I;
(ii) the topoisomerase inhibitor is selected from the group consisting of SN-38, camptothecin, topotecan, irinotecan, belotecan, rubitecan, exatecan, deruxtecan (DXd), gimatecan, silatecan, idenoisoquinoline, a phenanthridine, and an indolocarbazole;
(iii) the anti-Trop-2 antibody component of the ADC is sacituzumab (hRS7) or datopotamab;
(iv) the ADC comprises an anti-Trop-2 hRS7 antibody conjugated to an SN-38 topoisomerase inhibitor via a CL2A linker; and/or
(v) the ADC is sacituzumab govitecan or datopotamab deruxtecan (DS-1062).

3.-6. (canceled)

7. The method of claim 1, wherein the ADC is sacituzumab govitecan.

8. The method of claim 1, wherein the PARPi is selected from the group consisting of olaparib, talazoparib, rucaparib, veliparib, niraparib, pamiparib, CEP 9722, E7016, CEP-8983, and 3-aminobenzamide.

9. The method of claim 8, wherein the PARPi is talazoparib.

10. The method of claim 1, wherein the staggered dosing schedule comprises a 21-day cycle.

11. The method of claim 10, wherein the anti-Trop-2 ADC is administered on days 1 and 8 of the 21-day cycle.

12. The method of claim 10, wherein the PARPi is administered on days 15 to 21 of the 21-day cycle.

13. The method of claim 1, wherein the ADC is administered at a dosage of 8 mg/kg to 10 mg/kg.

14. The method of claim 1, wherein the ADC is administered at a dosage of 10 mg/kg.

15. The method of claim 1, wherein the PARPi is administered at a dosage of 0.5 mg/kg to 1.0 mg/kg.

16. The method of claim 1, wherein the PARPi is administered at a dosage of 1.0 mg/kg.

17. The method of claim 1, wherein the cancer is selected from the group consisting of colon cancer, stomach cancer, esophageal cancer, medullary thyroid cancer, kidney cancer, breast cancer, lung cancer, pancreatic cancer, urothelial cancer, ovarian cancer, uterine cancer, cervical cancer, testicular cancer, prostate cancer, liver cancer, skin cancer, bone cancer, head and neck cancer, brain cancer, glioblastoma, rectal cancer, and melanoma.

18. The method of claim 1, wherein:
(i) the cancer is selected from the group consisting of triple-negative breast cancer, HR+/HER2-breast cancer, HER2-low breast cancer, ovarian cancer, endometrial cancer, urothelial cancer, non-small-cell lung cancer, small-cell lung cancer and colorectal cancer;
(ii) the subject does not exhibit a mutation in BRCA1 or BRCA2; and/or
(iii) the cancer is metastatic.

19-20. (canceled)

21. The method of claim 1, wherein the cancer is HER2-negative metastatic breast cancer with deleterious or suspected deleterious germline breast cancer susceptibility gene (BRCA)-mutated (gBRCAm).

22. The method of claim 1, wherein the cancer is metastatic triple negative breast cancer (mTNBC).

23. The method of claim 1, wherein:
(i) the staggered dosing schedule reduces the toxicity of the treatment to normal tissues;
(ii) the staggered dosing schedule reduces the incidence of neutropenia; and/or
(iii) the staggered dosing schedule reduces the incidence of adverse events.

24-25. (canceled)

26. The method of claim 23, wherein:
(i) the adverse event is selected from the group consisting of neutropenia, anemia, thrombocytopenia, nausea, and diarrhea; or
(ii) the adverse events comprise severe myelosuppression.

27-28. (canceled)

29. The method of claim 1, wherein:
(i) the staggered dosing schedule improves overall survival relative to a continuous dosing schedule;
(ii) the cancer is metastatic and the treatment reduces in size or eliminates the metastases;
(iii) the cancer is refractory to at least one other therapy but responds to the combination of ADC and PARPi; and/or
(iv) the method further comprises administering to the subject one or more additional therapeutic modalities selected from the group consisting of unconjugated antibodies, radiolabeled antibodies, drug-conjugated antibodies, toxin-conjugated antibodies, gene therapy, chemotherapy, therapeutic peptides, cytokine therapy, oligonucleotides, localized radiation therapy, surgery and interference RNA therapy.

30-32. (canceled)

33. The method of claim 29, wherein the therapeutic modality comprises treatment with an agent selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celebrex, chlorambucil, cisplatin (CDDP), Cox-2 inhibitors, irinotecan (CPT-11), SN-38, carboplatin, cladribine, camptothecans, cyclophosphamide, crizotinib, cytarabine, dacarbazine, dasatinib, dinaciclib, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, flavopiridol, floxuridine (FUdR), 3′,5′-O-dioleoyl-FudR (FUdR-dO), fludarabine, flutamide, farnesyl-protein transferase inhibitors, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD1839.

34. A method of treating metastatic TNBC cancer, comprising administering sacituzumab govitecan and talazoparib to a human metastatic TNBC patient on a staggered dosing schedule, wherein sacituzumab govitecan is administered on days 1 and 8 of a 21 day cycle at a dosage of 8 mg/kg to 10 mg/kg, and talazoparib is administered on days 15 to 21 of the 21 day cycle at a dosage of 0.5 mg/kg to 1.0 mg/kg.

35. The method of claim 34, wherein sacituzumab govitecan is administered at a dosage of 8 mg/kg or 10 mg/kg.

36. (canceled)

37. The method of claim 34, wherein talazoparib is administered at a dosage of 0.5 mg/kg or 1.0 mg/kg.

38-40. (canceled)
